(19) United States
(12) Patent Application Publication
(10) Pub. No.: US 2021/0054372 A1
(43) Pub. Date: Feb. 25, 2021

(54) METHODS OF INACTIVATING GENE EDITING MACHINERIES

(71) Applicant: The Trustees of Columbia University in the City of New York, New York, NY (US)

(72) Inventors: Stephen H. Tsang, New York, NY (US); Wen-Hsuan Wu, New York, NY (US); Yi-Ting Tsai, New York, NY (US)

(21) Appl. No.: 17/074,021

(22) Filed: Oct. 19, 2020

Related U.S. Application Data
(63) Continuation of application No. PCT/US2019/029650, filed on Apr. 29, 2019.
(60) Provisional application No. 62/663,328, filed on Apr. 27, 2018.

(51) Int. Cl. C12N 15/11; C12N 9/22; C12N 15/90; A61K 38/46; A61K 31/7088

(52) U.S. Cl. CPC C12N 15/11; C12N 9/22; C12N 15/907; C12N 2800/80; A61K 31/7088; C12N 2310/20; A61K 38/465

(57) ABSTRACT

The present disclosure provides for systems and methods to limit the duration of a gene editing machinery, such as an endonuclease system. A self-terminating mechanism may be introduced by placing one or more target sequences (targeted by the gene editing machinery) on a polynucleotide encoding at least one component of the gene editing machinery/system.

### CROSS REFERENCE TO RELATED APPLICATION

This application is a continuation of International Patent Application No. PCT/US2019/029650, with an international filing date of Apr. 29, 2019, which claims priority to U.S. Provisional Application No. 62/663,328 filed on Apr. 27, 2018, all of which are incorporated by reference, as if expressly set forth in their respective entireties herein.

### SEQUENCE LISTING

The instant application contains a Sequence Listing which has been submitted electronically in ASCII format and is hereby incorporated by reference in its entirety. Said ASCII copy, created on Apr. 29, 2019, is named 01001_006743_WO0_ST25.txt and is 9 kilobytes in size.

### FIELD OF THE INVENTION

The present disclosure relates to systems and methods to self-regulate gene editing machineries.

### BACKGROUND OF THE DISCLOSURE

Gene editing machineries, such as RNA-guided endonucleases (RGENs), can be used to treat genetic disorders. The Clustered Regularly Interspaced Short Palindromic Repeats (CRISPR)-Cas system is a prokaryotic immune system that confers resistance to foreign genetic elements such as plasmids and bacteriophages. The CRISPR/Cas9 system exploits RNA-guided DNA-binding and sequence-specific cleavage of a target DNA. A guide RNA (gRNA) can be complementary to a target DNA sequence upstream of a PAM (protospacer adjacent motif) site. The Cas (CRISPR-associated) 9 protein binds to the gRNA and the target DNA and introduces a double-strand break (DSB) in a defined location upstream of the PAM site. Geurts et al., Science 325, 433 (2009); Mashimo et al., PLoS ONE 5, e8870 (2010); Carbery et al., Genetics 186, 451-459 (2010); Tesson et al., Nat. Biotech. 29, 695-696 (2011). Wiedenheft et al. Nature 482, 331-338 (2012); Jinek et al. Science 337, 816-821 (2012); Mali et al. Science 339, 823-826 (2013); Cong et al. Science 339, 819-823 (2013). The ability of the CRISPR/Cas9 system to be programmed to cleave not only viral DNA but also other genes opened a new venue for genome engineering. The CRISPR/Cas system has also been used for gene regulation including transcription repression and activation without altering the target sequence.

However, long-term expression of gene editing machineries in the cells can be genotoxic, as it may create unwanted, excessive DNA modification and potentially elicit undesirable immune responses. There remains a need for regulating and controlling gene editing machineries.

### SUMMARY

The present disclosure provides for a polynucleotide (e.g., a vector) comprising: (a) at least one gene encoding at least one component of an endonuclease system; and (b) a target sequence (e.g., a first target sequence) targeted by the endonuclease system.

The endonuclease system may comprise a CRISPR/Cas system.

The endonuclease system may comprise a transcription activator-like effector nuclease (TALEN), a zinc finger nuclease (ZFN), a ZFN dimer, or a ZFNickase.

The at least one gene may encode an RNA-guided DNA endonuclease, such as a Cas enzyme or a variant thereof. In one embodiment, the Cas enzyme is Cas9. In another embodiment, the Cas enzyme is a Cas nickase. In yet another embodiment, the Cas enzyme is a nuclease-defective Cas (dCas). The dCas may be fused to a repressor domain.

The at least one gene may encode a guide RNA (gRNA), a CRISPR RNA (crRNA), a trans-activating CRISPR RNA (tracrRNA), and/or a single-guide RNA (sgRNA).

The (first) target sequence may be within the at least one gene. The (first) target sequence may be outside the at least one gene.

The polynucleotide (e.g., a vector) may comprise 1, 2, 3, 4, 5, 6, 7, 8, 9, 10 or more genes that encode 1, 2, 3, 4, 5, 6, 7, 8, 9, 10 or more components of an endonuclease system.

The (first) target sequence may be flanked by a protospacer adjacent motif (PAM), e.g., a first PAM. The (first) PAM may be mutated or suboptimal. In one embodiment, the first PAM may comprise a nucleotide sequence NAG or a nucleotide sequence NGA.

In certain embodiments, there may be at least one mismatch (e.g., 1, 2, 3, 4, 5, 6, 7, 8, 9, 10, 11, 12, 13, 14, 15, 16, 17, 18, 19, 20 or more mismatches) between the (first) target sequence and the gRNA (or crRNA or sgRNA).

The (first) target sequence may be derived from a disease-related gene selected from the group consisting of, BEST1, PRDM13, RGR, TEAD1, AIPL1, CRX, GUCA1A, GUCY2D, PITPNM3, PROM1, PRPH2, RIMS1, SEMA4A, UNC119, GNAT1, PDE6B, RHO, WSF1, IMPDH1, OTX2, BEST1, C1QTNF5, CTNNA1, EFEMP1, ELOVL4, FSCN2, GUCA1B, HMCN1, IMPG1, RP1L1, TIMP3, VCAN, MFN2, NR2F1, OPA1, ARL3, CA4, HK1, KLHL7, NR2E3, NRL, PRPF3, PRPF4, PRPF6, PRPF8, PRPF31, RDH12, ROM1, RP1, RP9, RPE65, SNRNP200, SPP2, TOPORS, ABCC6, ATXN7, COL11A1, COL2A1, JAG1, KCNJ13, KIF11, OPA3, PAX2, TREX1, CAPNS, CRB1, FZD4, ITM2B, LRPS, MAPKAPK3, MIR204, OPN1SW, RB1, TSPAN12, and ZNF408.

The (first) target sequence may be derived from a tumor suppressor gene.

The present disclosure provides for a system comprising the present polynucleotide.

The present disclosure provides for a composition comprising the present polynucleotide or the present system.

The present disclosure provides for a cell comprising the present polynucleotide or the present system.

The present disclosure provides for a vector comprising the present polynucleotide or the present system. In one embodiment, the vector may be a recombinant adeno-associated viral (AAV) vector, such as AAV2, AAV8, or any other suitable type of AAV vector.

Also encompassed by the present disclosure is a method for inactivating an endonuclease system in a cell or in a subject. The method may comprise contacting a cell with the present polynucleotide, vector system, or composition. The method may comprise administering to the subject the present polynucleotide, vector, system, or composition.

The present disclosure provides for a method for modifying gene expression in a cell or in a subject. The method may comprise contacting a cell with the present polynucleotide, vector, system, or composition. The method may comprise administering to the subject the present polynucleotide, vector system, or composition.

The present disclosure provides for a method of treating a condition in a subject. The method may comprise administering to the subject the present polynucleotide, vector, system, or composition.

The cell or the subject may comprise a second target sequence targeted by the endonuclease system. The second target sequence may be flanked by a second protospacer adjacent motif (PAM). In one embodiment, the second PAM is wildtype. In one embodiment, the second PAM may comprise a nucleotide sequence NGG.

The (second) target sequence may be derived from a disease-related gene selected from the group consisting of, BEST1, PRDM13, RGR, TEAD1, AIPL1, CRX, GUCA1A, GUCY2D, PITPNM3, PROM1, PRPH2, RIMS1, SEMA4A, UNC119, GNAT1, PDE6B, RHO, WSF1, IMPDH1, OTX2, BEST1, C1QTNF5, CTNNA1, EFEMP1, ELOVL4, FSCN2, GUCA1B, HMCN1, IMPG1, RP1L1, TIMP3, VCAN, MFN2, NR2F1, OPA1, ARL3, CA4, HK1, KLHL7, NR2E3, NRL, PRPF3, PRPF4, PRPF6, PRPF8, PRPF31, RDH12, ROM1, RP1, RP9, RPE65, SNRNP200, SPP2, TOPORS, ABCC6, ATXN7, COL11A1, COL2A1, JAG1, KCNJ13, KIF11, OPA3, PAX2, TREX1, CAPN5, CRB1, FZD4, ITM2B, LRP5, MAPKAPK3, MIR204, OPN1SW, RB1, TSPAN12, and ZNF408.

The second target sequence may be derived from a tumor suppressor gene.

The second target sequence and the first target sequence may be derived from the same disease-related gene.

The second target sequence and the first target sequence may be identical or different. In certain embodiments, there may be no mismatch between the second target sequence and the gRNA (or crRNA or sgRNA), or the targeting segment of the gRNA (or crRNA, or sgRNA). In certain embodiments, there may be at least or about 1, 2, 3, 4, 5, 6, 7, 8, 9, 10, 11, 12, 13, 14, 15, 16, 17, 18, 19, 20 fewer mismatches between the second target sequence and the gRNA (or crRNA or sgRNA) than between the first target sequence and the gRNA (or crRNA or sgRNA).

The cell may be an induced pluripotent stem cell (iPSC), e.g., derived from a fibroblast of a subject.

The present method may further comprise culturing the iPSC to differentiate into a retinal pigment epithelium (RPE) cell.

The present method may further comprise administering the RPE cell to a subject.

The cell (e.g., the RPE cell) may be administered via subretinal transplantation.

The condition may be an ocular disease. Non-limiting examples of the ocular diseases include: age-related macular degeneration, juvenile macular degeneration, vitelliform macular dystrophy (VMD), Best vitelliform macular dystrophy, autosomal dominant chorioretinal atrophy or degeneration, autosomal dominant cone or cone-rod dystrophy, autosomal dominant congenital stationary night blindness, autosomal dominant Leber congenital amaurosis, autosomal dominant macular degeneration, autosomal dominant ocular-retinal developmental disease, autosomal dominant optic atrophy, autosomal dominant retinitis pigmentosa, autosomal dominant syndromic/systemic diseases with retinopathy, sorsby macular dystrophy, age-related macular degeneration, doyne honeycomb macular disease, juvenile macular degeneration, and combinations thereof.

The composition may be administered by injection into an eye of the subject.

### BRIEF DESCRIPTION OF THE DRAWINGS

FIG. 1 shows a scheme of an embodiment of the present system. Pro-Nu: Programmable nuclease that can be used to target a specific sequence (e.g., CRISPR-Cas systems, RGENs, TALENs and ZFNs).

FIGS. 2A-2D show one or more chosen sequence(s) to be targeted is/are placed near and/or inside the Pro-Nu expressing cassette. FIG. 2A shows that one or more chosen sequence(s) (e.g., one) to be targeted is placed near the Pro-Nu expressing cassette. Pro-Nu expressing cassette: Necessary components for gene expression (e.g., promoter, open reading frame and poly A tail). FIG. 2B shows that one or more chosen sequence(s) (e.g., two) to be targeted is placed near the Pro-Nu expressing cassette. FIG. 2C shows that one or more chosen sequence(s) to be targeted is placed inside the Pro-Nu expressing cassette. FIG. 2D shows that one or more chosen sequence(s) to be targeted are placed both near and inside the Pro-Nu expressing cassette.

FIG. 3 shows a scheme of a self-terminating system. In the case that another priority target exists (e.g., a gene to be destroyed other than the Pro-Nu gene), the designated Pro-Nu targeting sequence(s) on the vector expressing Pro-Nu can be different to that of the priority target (e.g., including mismatch(es) to the Pro-Nu targeting sequence, and/or suboptimal PAM) to ensure the destruction of the priority target before self-termination of Pro-Nu.

FIG. 4 shows an example of a Pro-Nu expressing cassette. In this case, genomic locus of rhodopsin exon 1 is shown.

FIGS. 5A-5D. FIG. 5A shows a scheme of a construct plasmid. FIGS. 5B-5D show CAG-Cas9-sg050-AGG (a target sequence followed by canonical PAM; FIG. 5B), CAG-Cas9-sg050-AAG (a target sequence followed by suboptimal PAM; FIG. 5C), and CAG-Cas9-sg050-AGA (a target sequence followed by suboptimal PAM; FIG. 5D) which contain the target sequence of human bestrophin 1, with different PAM sites respectively.

FIGS. 6A-6B show a scheme (FIG. 6A) and sequences (FIG. 6B) for plasmid PBS246 which contains the gRNA sequence targeting the human bestrophin 1 gene.

FIGS. 7A-7B show Western blotting results demonstrating the decreased level of Cas9. FIG. 7A shows Cas9 Western blotting results. FIG. 7B shows GFP Western blotting results. Lane 1. CAG-Cas9 plasmid. Lane 2. PBS246-sg050 (gRNA). Lane 3. PBS246-sg050 (gRNA)+CAG-Cas9-sg050 (target sequence)-AGG PAM. Lane 4. PBS246-sg050 (gRNA)+CAG-Cas9-sg050 (target sequence)-AGA (suboptimal PAM). Lane 5. PBS246-sg050 (gRNA)+CAG-Cas9-sg050 (target sequence)-AAG (suboptimal PAM).

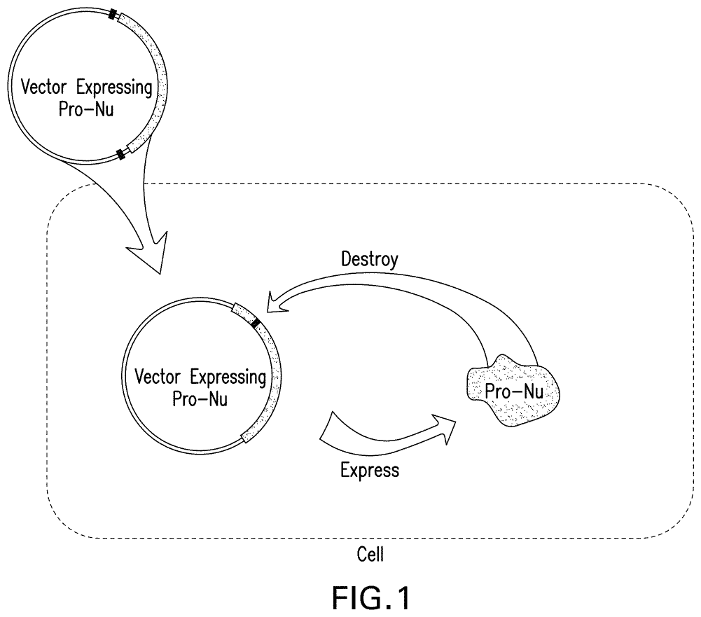

### DETAILED DESCRIPTION

The present disclosure provides for systems and methods to limit the duration of a gene editing machinery/system, such as an endonuclease system (e.g., a sequence-specific endonuclease). A self-terminating mechanism may be introduced by placing one or more target sequences (targeted by the gene editing machinery/system) on a polynucleotide (e.g., a vector) encoding at least one component of the gene editing machinery/system (e.g., an endonuclease system). When the gene editing machinery/system (e.g., the endonuclease system) is expressed, the polynucleotide (e.g., the vector) having the target sequence(s) can then be targeted by the gene editing machinery/system and thus inactivated or repressed. This self-inactivation can limit the potential toxicity conferred by the gene editing machinery/system. The present system/method may be used to terminate toxic, excessive gene editing when treating genetic diseases. The present system/method may reduce toxicity and/or immunogenicity caused by, or associated with, the gene editing machinery.

When the gene editing machinery/system (e.g., the endonuclease system) is used to target a target sequence (e.g., a second target sequence) in a cell (e.g., in a cellular gene to be targeted (e.g., cleaved or repressed) other than the gene encoding the gene editing machinery/system), strategies may be used to ensure that the gene editing machinery/system is self-terminated after the cellular target sequence (e.g., a second target sequence) is targeted by the gene editing machinery/system. Such strategies may include introducing one or more mismatches into the first target sequence on the polynucleotide (e.g., a vector) (e.g., one or more mismatches between the first target sequence and the gRNA), and using suboptimal PAM for the first target sequence on the polynucleotide (e.g., a vector).

The present method/system may be used to minimize the intensity and duration of the endonuclease system expression and potential off-targeting effects, where the endonuclease system expression/activity is self-inactivated or decreased after production. It is anticipated that the amount/activity of one or more component of the endonuclease system present (before the self-termination) is sufficient to target the desired cellular locus. The self-inactivating mechanism can control the amount and duration of the endonuclease system expression in target cells, and can prevent or decrease the unwanted off-target effects due to excessive expression of the endonuclease system.

The present disclosure provides for a polynucleotide comprising: (a) at least one gene encoding at least one component of an endonuclease system; and (b) a first target sequence targeted/recognized by the endonuclease system.

The target sequence(s) can be placed within and/or outside (e.g., near, around, or adjacent to) the gene encoding at least one component of the gene editing machinery/system.

The target sequence on the polynucleotide (e.g., a first target sequence) may be within the at least one gene. The first target sequence may be outside (e.g., adjacent to) the at least one gene, for example, about 1 nucleotide, 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, or more apart from the at least one gene.

The endonuclease system may cleave the first target sequence, and/or may repress/inhibit transcription/expression of the at least one component of an endonuclease system (with or without altering the target sequence).

Also encompassed by the present disclosure is a system comprising the present polynucleotide. In one embodiment, the polynucleotide (e.g., a vector) comprises a gene encoding a Cas or a variant thereof and a gene encoding a gRNA (or crRNA, or sgRNA). In another embodiment, the polynucleotide (e.g., a first polynucleotide) comprises a gene encoding a Cas or a variant thereof. The system may further comprise a second polynucleotide comprising a gene encoding a gRNA (or crRNA, or sgRNA), or further comprise a gRNA (or crRNA, or sgRNA). In yet another embodiment, the polynucleotide (e.g., a first polynucleotide) comprises a gene encoding a gRNA (or crRNA, or sgRNA). The system may further comprise a second polynucleotide comprising a gene encoding a Cas or a variant thereof, or further comprise a Cas or a variant thereof.

The endonuclease system may comprise a transcription activator-like effector nuclease (TALEN), a zinc finger nuclease (ZFN), a ZFN dimer, or a ZFNickase.

The target sequence may be on the polynucleotide (e.g., the vector) expressing Cas and/or gRNA (or crRNA, or sgRNA) to enable self-termination of the CRISPR/Cas system.

For example, the at least one gene may encode an RNA-guided DNA endonuclease, such as a Cas enzyme or a variant thereof. The at least one gene may encode a guide RNA (gRNA), a CRISPR RNA (crRNA), a trans-activating CRISPR RNA (tracrRNA), and/or a single-guide RNA (sgRNA).

The target sequence on the polynucleotide (e.g., a first target sequence) may be flanked by a protospacer adjacent motif (PAM) (e.g., a first PAM). In certain embodiments, the PAM (e.g., the first PAM) may be mutated or suboptimal for the Cas enzyme or a variant thereof. In certain embodiments, the first PAM may be wildtype or optimal for the Cas enzyme or a variant thereof.

In one embodiment, there is one or more mismatches between the target sequence on the polynucleotide (e.g., the first target sequence) and the gRNA, crRNA, or sgRNA.

The present disclosure provides for a method of inactivating an endonuclease system. The method may comprise introducing into a cell the present polynucleotide.

The present disclosure provides for a method of modifying a disease-related gene. The method may comprise introducing into a cell the present polynucleotide.

The cell may comprise a second target sequence targeted by the endonuclease system. The second target sequence may be flanked by a second protospacer adjacent motif (PAM). In certain embodiments, the second PAM may be wildtype or optimal for the Cas enzyme or a variant thereof.

In certain embodiments, the PAM (e.g., the first PAM) flanking the first target sequence on the polynucleotide (e.g., a vector) may be mutated or suboptimal for the Cas enzyme or a variant thereof. The PAM flanking the cellular target sequence (e.g., the second target sequence) may be wildtype or optimal for the Cas enzyme or a variant thereof.

In certain embodiments, there are one or more mismatches between the target sequence on the polynucleotide (e.g., the first target sequence) and (the targeting segment of) the gRNA, crRNA, or sgRNA. There may be no mismatch between the cellular target sequence (e.g., the second target sequence) and (the targeting segment of) the gRNA, crRNA, or sgRNA. In one embodiment, there is/are 1, 2, 3, 4, 5, 6, 7, 8, 9, 10, 11, 12, 13, 14, 15, etc. more mismatches (mismatched nucleotides) between the first target sequence on the polynucleotide (e.g., the vector) and (the targeting segment of) the guide RNA (gRNA) (or crRNA, or sgRNA) than the number of mismatches between the cellular target sequence (e.g., the second target sequence) and (the targeting segment of) the guide RNA (gRNA) (or crRNA, or sgRNA).

The present system/method may apply to any suitable gene editing machinery/system. Non-limiting examples of the endonucleases include a zinc finger nuclease (ZFN), a ZFN dimer, a ZFNickase, a transcription activator-like effector nuclease (TALEN), or an RNA-guided endonuclease (RGEN) (e.g., an RNA-guided DNA endonuclease, such as the CRISPR/Cas system). Meganucleases are endonucleases characterized by their capacity to recognize and cut large DNA sequences (12 base pairs or greater). Any suitable meganuclease may be used in the present system/method, such as endonucleases in the LAGLIDADG family.

The Cas/CRISPR system exploits RNA-guided DNA-binding and sequence-specific cleavage of a target DNA. A guide RNA (gRNA) are complementary to a target DNA sequence that may be upstream of a PAM (protospacer adjacent motif) site. The Cas (CRISPR-associated) protein binds to the gRNA and the target DNA and introduces a cut in a defined location that may be upstream of the PAM site. Geurts et al., Science 325, 433 (2009); Mashimo et al., PLoS ONE 5, e8870 (2010); Carbery et al., Genetics 186, 451-459 (2010); Tesson et al., Nat. Biotech. 29, 695-696 (2011). Wiedenheft et al. Nature 482, 331-338 (2012); Jinek et al. Science 337, 816-821 (2012); Mali et al. Science 339, 823-826 (2013); Cong et al. Science 339, 819-823 (2013).

The endonuclease system may comprise an RNA-guided DNA endonuclease, such as a Cas enzyme or a variant thereof, a guide RNA (gRNA), a CRISPR RNA (crRNA), a single-guide RNA (sgRNA), or combinations thereof. The endonuclease system may comprise a CRISPR/Cas system.

In one embodiment, the present system/method provides an endonuclease (e.g., Cas) mediated cleavage event in the nucleic acid sequence that encodes at least one component of an endonuclease system (e.g., Cas and/or gRNA, or crRNA, or sgRNA). In an embodiment, the present system/method inactivates or silences a polynucleotide (a nucleic acid) that comprises a gene encoding the Cas molecule. In an embodiment, the present system/method inactivates or silences a polynucleotide (a nucleic acid) that comprises a gene encoding a gRNA (or crRNA, or sgRNA). In an embodiment, a target sequence is located within, and/or near (adjacent to), a gene that encodes Cas. In an embodiment, a target sequence is located within, and/or near (adjacent to), a gene that encodes a gRNA (or crRNA, or sgRNA).

The present system/method may edit (e.g., cleave), inactivate, silence, and/or repress, at least one component of an endonuclease system. In an embodiment, the present system/method limits the effect of the endonuclease system-mediated gene targeting. In an embodiment, the present system/method places temporal, level of expression, or other limits, on activity of the endonuclease system. In an embodiment, the present system/method reduces off-target or other unwanted activity of the endonuclease system. The present system/method may inhibit, e.g., entirely or substantially, the production of at least one component of the endonuclease system, and thereby inhibit, limit, or decrease its activity.

In an embodiment, the nucleic acid sequence encoding at least one component of an endonuclease system has been cleaved. In an embodiment, the nucleic acid sequence encoding at least one component of an endonuclease system has not been cleaved. In an embodiment, the nucleic acid sequence encoding at least one component of an endonuclease system can express at a decreased level compared to a polynucleotide without the target sequence (e.g., the first target sequence).

In an embodiment, the polynucleotide is substantially incapable of expressing the at least one component of an endonuclease system. In an embodiment, the polynucleotide does not express the at least one component of an endonuclease system.

The present system/method may result in termination of the expression or activity of at least one component of the endonuclease system, or may result in a reduction in the expression level or activity of at least one component of the endonuclease system, by at least or about 10%, at least or about 15%, at least or about 20%, at least or about 25%, at least or about 30%, at least or about 35%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 91%, at least or about 92%, at least or about 93%, at least or about 94%, at least or about 95%, at least or about 96%, at least or about 97%, at least or about 98%, or at least or about 99%, in about 2 hours, in about 5 hours, in about 10 hours, in about 24 hours, in about 1 day, in about 2 days, in about 3 days, in about 4 days, in about 5 days, in about 6 days, in about 1 week, in about 2 weeks, in about 3 weeks, in about 4 weeks, in about 5 weeks, in about 6 weeks, in about 7 weeks, in about 8 weeks, in about 9 weeks, in about 10 weeks, in about 11 weeks, in about 1 month, in about 2 months, in about 3 months, in about 4 months, in about 5 months, in about 6 months, from about 1 week to about 2 weeks, or within different time-frames following administration to a subject and/or cells (or contacting the cells).

The expression level and/or activity of the at least one component of an endonuclease system may decrease by about 1% to about 100%, about 5% to about 90%, about 10% to about 80%, about 5% to about 70%, about 5% to about 60%, about 10% to about 50%, about 15% to about 40%, about 5% to about 20%, about 1% to about 20%, about 10% to about 30%, at least or about 5%, at least or about 10%, at least or about 15%, at least or about 20%, at least or about 30%, at least or about 40%, at least or about 50%, at least or about 60%, at least or about 70%, at least or about 80%, at least or about 90%, at least or about 100%, about 10% to about 90%, about 12.5% to about 80%, about 20% to about 70%, about 25% to about 60%, or about 25% to about 50%, at least or about 2 fold, at least or about 3 fold, at least or about 4 fold, at least or about 5 fold, at least or about 6 fold, at least or about 7 fold, at least or about 8 fold, at least or about 9 fold, at least or about 10 fold, at least or about 1.5 fold, at least or about 2.5 fold, at least or about 3.5 fold, at least or about 15 fold, at least or about 20 fold, at least or about 50 fold, at least or about 100 fold, at least or about 120 fold, from about 2 fold to about 500 fold, from about 1.1 fold to about 10 fold, from about 1.1 fold to about 5 fold, from about 1.5 fold to about 5 fold, from about 2 fold to about 5 fold, from about 3 fold to about 4 fold, from about 5 fold to about 10 fold, from about 5 fold to about 200 fold, from about 10 fold to about 150 fold, from about 10 fold to about 20 fold, from about 20 fold to about 150 fold, from about 20 fold to about 50 fold, from about 30 fold to about 150 fold, from about 50 fold to about 100 fold, from about 70 fold to about 150 fold, from about 100 fold to about 150 fold, from about 10 fold to about 100 fold, from about 100 fold to about 200 fold, compared to a polynucleotide without the target sequence (e.g., the first target sequence), in about 2 hours, in about 5 hours, in about 10 hours, in about 24 hours, in about 1 day, in about 2 days, in about 3 days, in about 4 days, in about 5 days, in about 6 days, in about 1 week, in about 2 weeks, in about 3 weeks, in about 4 weeks, in about 5 weeks, in about 6 weeks, in about 7 weeks, in about 8 weeks, in about 9 weeks, in about 10 weeks, in about 11 weeks, in about 1 month, in about 2 months, in about 3 months, in about 4 months, in about 5 months, in about 6 months, from about 1 week to about 2 weeks, or within different time-frames following administration to a subject and/or cells (or contacting the cells).

The present system/method can target any region of the nucleic acid that comprises the gene encoding the component to be negatively regulated, within or outside the transcribed or translated region of the gene, as long as production of the component is reduced.

In an embodiment, a target sequence is on the nucleic acid on which the sequence encoding the component to be negatively regulated (e.g., Cas, gRNA or sgRNA or crRNA) resides.

In certain embodiments, the polynucleotide or vector comprises a nucleotide sequence about 80% to about 100%, at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the nucleotide sequence (or identical to the complementary sequence of the nucleotide sequence) set forth in SEQ ID NO: 1, SEQ ID NO: 2, SEQ ID NO: 8, SEQ ID NO: 9, SEQ ID NO: 12, SEQ ID NO: 13 SEQ ID NO: 15, SEQ ID NO: 16, SEQ ID NO: 18, or SEQ ID NO: 19.

In certain embodiments, the target sequence (e.g., a first target sequence, a second target sequence, etc.) comprises a nucleotide sequence about 80% to about 100%, at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the nucleotide sequence (or identical to the complementary sequence of the nucleotide sequence) set forth in SEQ ID NO: 4, SEQ ID NO: 5, SEQ ID NO: 6, SEQ ID NO: 11, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, or SEQ ID NO: 34.

In certain embodiments, the guide RNA (or sgRNA or crRNA) comprises a nucleotide sequence about 80% to about 100%, at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the nucleotide sequence (or identical to the complementary sequence of the nucleotide sequence) set forth in SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, or SEQ ID NO: 34.

The Cas enzyme of the CRISPR/Cas system may be Cas9, Cas1, Cas1B, Cas2, Cas3, Cas4, Cas5, Cas6, Cas7, Cas8, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, homologs thereof, orthologs thereof, or modified versions thereof.

As an example, CRISPR/Cas may be encoded by a viral vector, e.g., for therapeutic use. The present system/method may include one or more target sequences on the polynucleotide (e.g., a vector) encoding Cas and/or gRNA to enable self-termination of the CRISPR/Cas system.

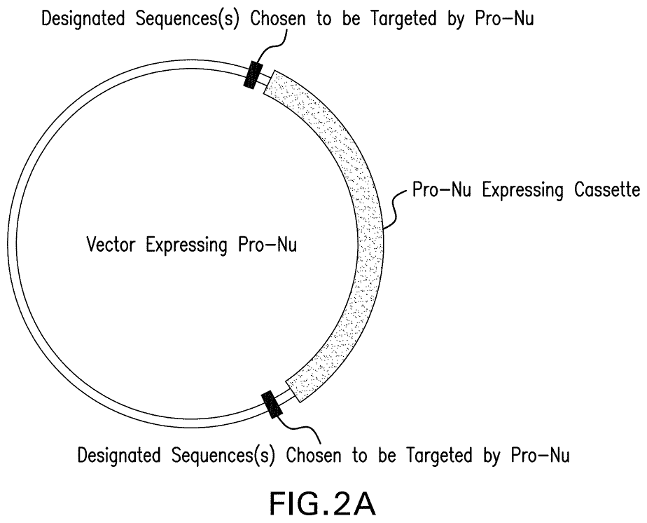

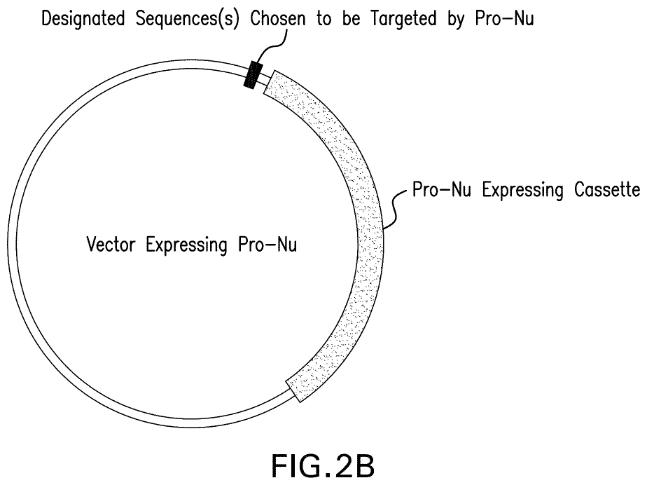

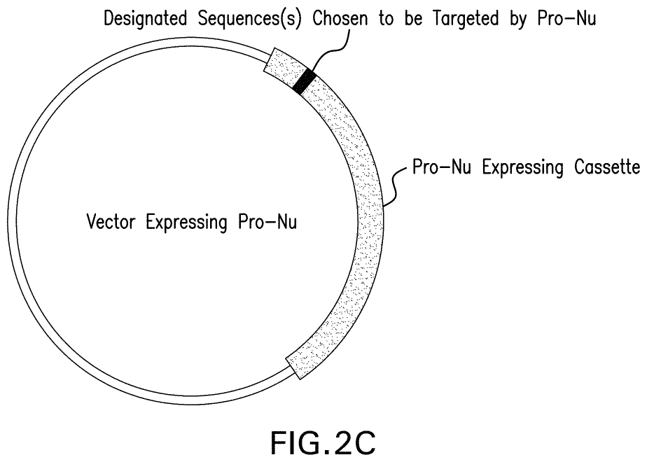

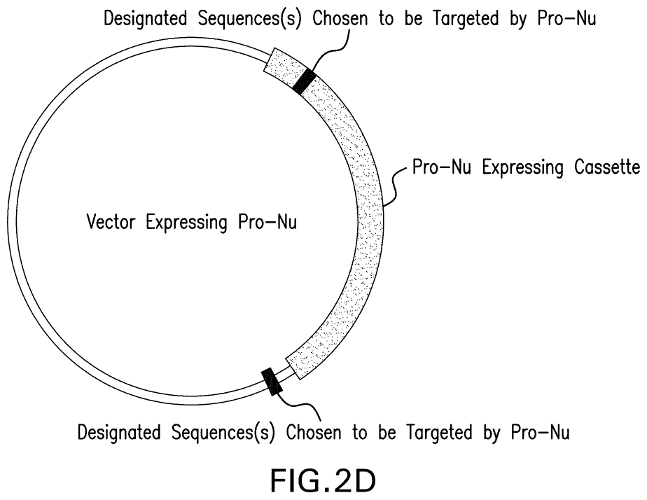

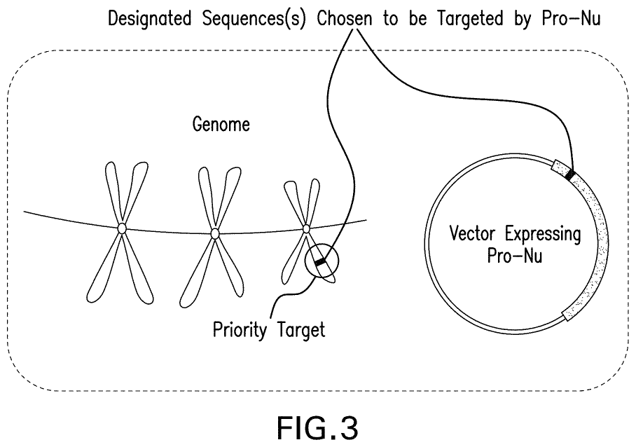

One or more target sequences (e.g., a first target sequence) on the polynucleotide (e.g., a vector) may be placed outside, e.g., near or adjacent to, the gene encoding the gene editing machinery/system (e.g., FIGS. 2A-2B). One or more target sequences (e.g., a first target sequence) on the polynucleotide (e.g., a vector) may be placed within the gene encoding the gene editing machinery/system (e.g., FIG. 2C). One or more target sequences (e.g., a first target sequence) on the polynucleotide (e.g., a vector) may be placed both outside and within the gene encoding the gene editing machinery/system (FIG. 2D).

The first target sequence(s) on the polynucleotide (e.g., the vector) encoding the gene editing machinery/system may be different from the second target sequence (e.g., in the cellular gene). The first target sequence on the polynucleotide (e.g., a vector) and the second target sequence (e.g., in the cellular gene) may be identical.

The gRNA (or crRNA, or sgRNA) may contain a targeting segment that can be fully complementary or substantially complementary (e.g., at least about 70% complementary (e.g., at least or about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a target sequence (“target region” or “target DNA”). In certain embodiments, the gRNA (or crRNA, or sgRNA) sequence (or the targeting segment of the gRNA (or crRNA, or sgRNA)) has 100% complementarity to the target sequence. The targeting segment of the gRNA (or crRNA, or sgRNA) may have full complementarity with the target sequence. The targeting segment of the gRNA (or crRNA, or sgRNA) may have partial complementarity with the target sequence. In certain embodiments, the targeting segment of the gRNA has or includes 1, 2, 3, 4, 5, 6, 7 or 8 nucleotides that are not complementary with the corresponding nucleotide of the target sequence (mismatches).

In one embodiment, there is/are one or more mismatches between the first target sequence on the polynucleotide (e.g., the vector) and the guide RNA (gRNA) (or crRNA, or sgRNA), such as 1, 2, 3, 4, 5, 6, 7, or more mismatches. The percent complementarity between the first target sequence and the gRNA (or crRNA, or sgRNA) may be about 60%, about 65%, about 70%, about 75%, about 80%, about 85%, about 90%, about 95%, about 97%, about 98%, about 99%, at least 60%, at least 65%, at least 70%, at least 75%, at least 80%, at least 85%, at least 90%, at least 95%, at least 97%, at least 98%, at least 99%, or about 100%.

In certain embodiments, the targeting segment of the gRNA (or crRNA, or sgRNA) is 16, 17, 18, 19, 20, 21, 22, 23, 24, 25, 26 nucleotides in length. In certain embodiment, the targeting segment of the gRNA (or crRNA, or sgRNA) is 10 to 100, 10 to 90, 10 to 80, 10 to 70, 10 to 60, 10 to 50, 10 to 40, 10 to 30, 10 to 20 or 10 to 15 nucleotides in length. In certain embodiments, the targeting segment of the gRNA (or crRNA, or sgRNA) is 20 to 100, 20 to 90, 20 to 80, 20 to 70, 20 to 60, 20 to 50, 20 to 40, 20 to 30, or 20 to 25 nucleotides in length.

In one embodiment, the degree of complementarity, together with other properties of the gRNA (or crRNA, or sgRNA), is sufficient to allow targeting of a Cas molecule to the target nucleic acid.

In some embodiments, a target sequence may be about 10 to about 40 consecutive nucleotides in length. A target sequence may be at least 10 consecutive nucleotides (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 consecutive nucleotides, and the like, and any range or value therein). In some embodiments, the target sequence can be about 10 to about 20 consecutive nucleotides, about 10 to about 30 consecutive nucleotides, and/or about 10 to about 40 consecutive nucleotides and the like, or any range or value therein.

In some embodiments, a target sequence (e.g., a first target sequence, a second target sequence) is located within an essential gene or a non-essential gene.

In an embodiment, the target sequence disclosed herein may be derived from a gene (e.g., a disease-related gene) described herein.

In an embodiment, the gene editing machinery/system (e.g., the endonuclease system) forms a single strand break in the target nucleic acid. In an embodiment, the single strand break is formed in the complementary strand of the target nucleic acid. In an embodiment, the single strand break is formed in the strand which is not the complementary strand of the target nucleic acid.

In an embodiment, the gene editing machinery/system (e.g., the endonuclease system) forms a double strand break in the target nucleic acid.

In an embodiment, the composition/system further comprises a second polynucleotide. The second polynucleotide may comprise Cas and/or a gRNA (or crRNA, or sgRNA). The second polynucleotide may or may not comprise the present target sequence (e.g., a first target sequence, or a third target sequence). The third target sequence and the first target sequence may be identical or may be different.

In one embodiment, the first polynucleotide may comprise a first gRNA; the second polynucleotide may comprise a second gRNA. Alternatively, a polynucleotide may comprise a first gRNA and a second gRNA. In an embodiment, the gRNA molecule and the second gRNA molecule mediate breaks at different sites in the target nucleic acid, e.g., flanking a target position. In an embodiment, the gRNA molecule and the second gRNA molecule are complementary to the same strand of the target. In an embodiment, the gRNA molecule and the second gRNA molecule are complementary to the different strands of the target. In an embodiment, the gRNA molecule and the second gRNA molecule are configured such that the first and second breaks made by the Cas molecule flank a target position. In an embodiment, the gRNA molecule and the second gRNA molecule are configured such that a first and second breaks are formed in the same strand of the nucleic acid target, e.g., in the case of transcribed sequence, the template strand or the non-template strand. In an embodiment, the first and second breaks flank a target position. In an embodiment, the gRNA molecule and the second gRNA molecule are configured such that a first and a second breaks are formed in different strands of the target. In an embodiment, the first and second breaks flank a target position.

A PAM site is a nucleotide sequence in proximity to a target sequence. For example, PAM may be a DNA sequence immediately following the DNA sequence targeted by the CRISPR/Cas system. The target sequence may or may not be flanked by a protospacer adjacent motif (PAM) sequence. In certain embodiments, a nucleic acid-guided nuclease can only cleave a target sequence if an appropriate PAM is present. Doudna et al., The new frontier of genome engineering with CRISPR/Cas9, Science, 2014, 346(6213): 1258096. A PAM can be 5′ or 3′ of a target sequence. A PAM can be upstream or downstream of a target sequence. In one embodiment, the target sequence is immediately flanked on the 3′ end by a PAM sequence. A PAM can be 1, 2, 3, 4, 5, 6, 7, 8, 9, 10 or more nucleotides in length. In certain embodiments, a PAM is between 2-6 nucleotides in length. The target sequence may or may not be located adjacent to a PAM sequence (e.g., PAM sequence located immediately 3′ of the target sequence) (e.g., for Type I CRISPR/Cas systems and Type II CRISPR/Cas systems). In some embodiments, e.g., Type I systems, the PAM is on the alternate side of the protospacer (the 5′ end). Makarova et al. describes the nomenclature for all the classes, types and subtypes of CRISPR systems (Nature Reviews Microbiology 13:722-736 (2015)). Guide structures and PAMs are described in by R. Barrangou (Genome Biol. 16:247 (2015)).

PAMs may be different between two different endonucleases. Cas9 isoforms derived from different species can display different PAM specificities. Esvelt et al., Orthogonal Cas9 proteins for RNA-guided gene regulation and editing, Nature Methods 10, 1116-1121 (2013). Zhang et al., Processing-independent CRISPR RNAs limit natural transformation in Neisseria meningitidis, Molecular Cell 50, 488-503 (2013). In an embodiment, the ability of a Cas9 molecule to interact with and cleave a target nucleic acid is PAM sequence dependent. In one embodiment, cleavage of the target nucleic acid occurs upstream from the PAM sequence. Cas9 molecules from different bacterial species may recognize different PAM sequences. A Cas9 molecule of S. pyogenes may recognize the sequence motif NGG and direct cleavage of a target nucleic acid sequence 1 to 10, e.g., 3 to 5, base pairs upstream from that sequence. See, e.g., Mali et al., SCIENCE 2013; 339(6121): 823-826. A Cas9 molecule of S. thermophilus may recognize the sequence motif NGGNG and NNAGAAW (W=A or T) and direct cleavage of a core target nucleic acid sequence 1 to 10, e.g., 3 to 5, base pairs upstream from these sequences. See, e.g., Horvath et al., SCIENCE 2010; 327(5962):167-170, and Deveau et al., J BACTERIOL 2008; 190(4): 1390-1400. A Cas9 molecule of S. mutans may recognize the sequence motif NGG or NAAR (R=A or G) and direct cleavage of a core target nucleic acid sequence 1 to 10, e.g., 3 to 5 base pairs, upstream from this sequence. See, e.g., Deveau et al., J BACTERIOL 2008; 190(4): 1390-1400. A Cas9 molecule of S. aureus may recognize the sequence motif NNGRR (R=A or G) and direct cleavage of a target nucleic acid sequence 1 to 10, e.g., 3 to 5, base pairs upstream from that sequence. A Cas9 molecule of N. meningitidis may recognize the sequence motif NNNNGATT and direct cleavage of a target nucleic acid sequence 1 to 10, e.g., 3 to 5, base pairs upstream from that sequence. See, e.g., Hou et al., PNAS early edition, 2013, 1-6.

The ability of a Cas9 molecule to recognize a PAM sequence can be determined, e.g., using a transformation assay described in Jinek et al., SCIENCE 2012, 337:816.

In one embodiment, the PAM flanking the first target sequence on the polynucleotide may be a suboptimal PAM. Suboptimal PAM (e.g., a mutated PAM, non-canonical PAM, modified PAM etc.) may have a lower/weaker binding affinity to the CRISPR/Cas system (e.g., Cas or its variant) than an optimal or wildtype PAM. The PAM mutation may be any insertion, deletion or substitution of one or more nucleotides (e.g., 1, 2, 3, 4, 5, 6, 7, 8, 9, 10 or more nucleotides) that mutates the sequence of the wildtype PAM such that it has a decreased affinity to the CRISPR system (e.g., Cas).

Non-limiting examples of suboptimal PAMs include GGA, NNNNGAAT, NAG, NGA, NGC, etc. Fu et al., High-frequency off-target mutagenesis induced by CRISPR-Cas nucleases in human cells. Nature Biotechnology 31, 822-826 (2013); Pattanayak et al., High-throughput profiling of off-target DNA cleavage reveals RNA-programmed Cas9 nuclease specificity. Nature Biotechnology 31, 839-843 (2013); and Hsu et al., DNA targeting specificity of RNA-guided Cas9 nucleases. Nature Biotechnology 31, 827-832 (2013). PAM can be any sequences disclosed in U.S. Pat. No. 10,190,106.

In another embodiment, the PAM flanking the first target sequence on the polynucleotide may be a non-canonical PAM, while the PAM flanking the second target sequence of the cellular gene may be a canonical PAM. For example, for the Cas9 nuclease of Streptococcus pyogenes, the canonical PAM is the sequence 5′-NGG-3′ where “N” is any nucleotide. Different PAMs are associated with the Cas9 proteins of other bacteria such as Neisseria meningitidis, Treponema denticola, and Streptococcus thermophilus. For example, non-canonical PAM may be the sequence 5′-NGA-3′ or 5′-NAG-3′.

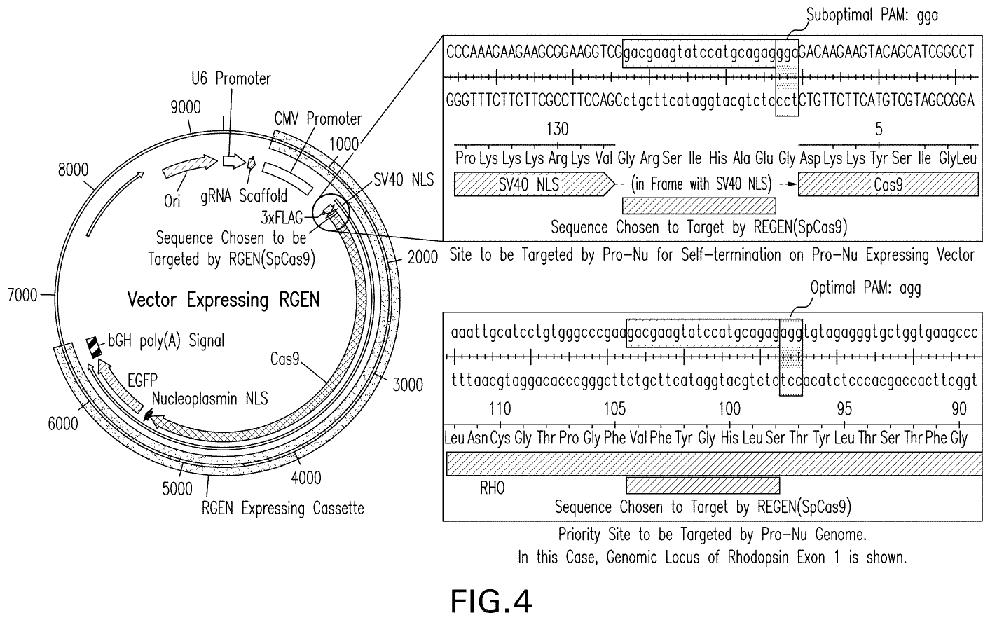

FIG. 4 shows examples of an optimal PAM and a suboptimal PAM. The upper right panel of FIG. 4 shows the site on the vector (e.g., a first target sequence) to be targeted by the CRISPR/Cas system for self-termination. In the lower right panel of FIG. 4, genomic locus of rhodopsin (RHO) exon 1 is shown as the targeted site in the genome (e.g., a second target sequence) to be targeted by the CRISPR/Cas system.

A PAM mutation can be a silent mutation. A silent mutation can be a change to at least one nucleotide of a codon relative to the original codon that does not change the amino acid encoded by the original codon. A silent mutation can be a change to a nucleotide within a non-coding region, such as an intron, 5′ untranslated region, 3′ untranslated region, or other non-coding region. A PAM mutation can be a non-silent mutation. Non-silent mutations can include a missense mutation. A missense mutation can be when a change to at least one nucleotide of a codon relative to the original codon that changes the amino acid encoded by the original codon. Missense mutations can occur within an exon, open reading frame, or other coding region.

When the endonuclease system comprises a TALEN, its TAL effector DNA binding domain may be engineered to recognize the first target sequence on the polynucleotide (e.g., a vector) and/or the second target sequence (e.g., in the cellular gene).

The present disclosure provides a cell comprising: a polypeptide described herein; a nucleic acid described herein; a vector described herein; or a composition described herein.

The cell may be a vertebrate, mammalian (e.g., human), rodent, goat, pig, bird, chicken, turkey, cow, horse, sheep, fish, or primate, cell. The cell may be a plant cell. In an embodiment, the cell is a human cell.

The cell may be a somatic cell, germ cell, or prenatal cell. The cell may be a zygotic, blastocyst or embryonic cell, a stem cell, a mitotically competent cell, a meiotically competent cell.

In an embodiment, the cell is a cancer cell or other cell characterized by a disease or disorder.

In an embodiment, the cell is a cell of a disease-causing organism, e.g., a virus, bacterium, fungus, protozoan, or parasite.

In an embodiment, the target sequence is derived from the nucleic acid of a disease-causing organism, e.g., a virus, bacterium, fungus, protozoan, or parasite.

In an embodiment, the target sequence is derived from the nucleic acid of a human cell. In an embodiment, the target sequence is derived from the nucleic acid of: a somatic cell, germ cell, prenatal cell, e.g., zygotic, blastocyst or embryonic, blastocyst cell, a stem cell, a mitotically competent cell, a meiotically competent cell.

In an embodiment, the target sequence is derived from a chromosomal nucleic acid. In an embodiment, the target sequence is derived from an organellar nucleic acid. In an embodiment, the target sequence is derived from a mitochondrial nucleic acid. In an embodiment, the target sequence is derived from a chloroplast nucleic acid.

In an embodiment, the cell is a cell characterized by unwanted proliferation, e.g., a cancer cell. In an embodiment, the cell is a cell characterized by an unwanted genomic component (e.g., a viral genomic component), such as a cell infected with viruses, a cell infected with bacteria etc.

In an embodiment, the target sequence is derived from the nucleic acid of a cell characterized by unwanted proliferation, e.g., a cancer cell. In an embodiment, the target sequence is derived from an unwanted genomic component, e.g., a viral genomic component. In an embodiment, the target sequence is derived from a rearrangement, e.g., a rearrangement that comprises a kinase gene, or a rearrangement that comprises a tumor suppressor gene. In an embodiment, the target sequence is derived from an oncogene (wildtype or mutant), a kinase gene (wildtype or mutant) or a tumor suppressor gene (wildtype or mutant).

In an embodiment, the gene editing machinery/system (e.g., the endonuclease system) targets a selected genomic signature, e.g., a mutation, such as a germline or acquired somatic mutation. In an embodiment, the gene editing machinery/system (e.g., the endonuclease system) targets a wildtype or mutant disease-related gene.

The present disclosure provides a pharmaceutical composition comprising: a polypeptide described herein; a nucleic acid described herein; a vector described herein, a system described herein, or a cell described herein.

The present disclosure provides a method of modulating the expression of a gene or inactivating a disease organism in a cell. The method may comprise contacting the cell with the present polynucleotide (nucleic acid), present system, or present composition.

In an aspect, the disclosure features a method of altering a cell, e.g., altering the structure, e.g., sequence, of a target nucleic acid of a cell, comprising contacting the cell with the present polynucleotide (nucleic acid), present system, or present composition.

The present disclosure provides a method of modifying a disease-related gene (wildtype or mutant) in a cell. The method may comprise contacting the cell with the present polynucleotide (nucleic acid), present system, or present composition.

In another aspect, the disclosure features a method of treating a subject, e.g., by altering the structure, e.g., altering the sequence, of a target nucleic acid (in a cell of the subject). The method may comprise administering to the subject (or contacting the cell of the subject), an effective amount of the polynucleotide (nucleic acid) described herein.

The present disclosure provides a method of treating a disease or condition in a subject. The method may comprise administering the present polynucleotide (nucleic acid), present composition, present system, or present cells to the subject.

In an embodiment, the subject is an animal or plant. In an embodiment, the subject is a mammalian, primate, or human.

In an embodiment, the cell comprises the target nucleic acid (e.g., the second target sequence) that has been cleaved, inhibited or repressed.

The present disclosure provides a reaction mixture comprising a cell and: a polypeptide described herein; a nucleic acid described herein; a vector described herein; a system described herein, or a composition described herein.

The present disclosure provides a kit comprising: a polypeptide described herein; a nucleic acid described herein; a vector described herein; a system described herein, or a composition described herein. The kit may comprise an instruction for using the polypeptide, the nucleic acid, the vector, or the composition, in a method described herein.

The present system/method may be used to treat macular degenerative and dominantly-inherited conditions.

The present disclosure provides for a method for modifying an autosomal dominant disease-related gene (e.g., an autosomal dominant ocular disease-related gene) in a cell.

The present disclosure provides for a method for modifying an autosomal dominant disease-related gene (e.g., an autosomal dominant ocular disease-related gene) in a cell.

The method may comprise contacting the cell with the present polynucleotide(s) or composition.

The cell may be from a subject having a dominant disease, such as an autosomal dominant ocular disease. The cell may be derived from a cell from a subject having a dominant disease condition, such as an autosomal dominant ocular disease.

The cell may be an induced pluripotent stem cell (iPSC), e.g., derived from a fibroblast of a subject.

The method may further comprise culturing the iPSC to differentiate into a retinal pigment epithelium (RPE) cell. The method may further comprise administering the RPE cell to a subject. In one embodiment, the RPE cell is administered via subretinal transplantation.

The RPE cell may be autologous or allogeneic to the subject.

The present disclosure provides for a method for treating an autosomal dominant disease (e.g., an autosomal dominant ocular disease) in a subject. The method may comprise administering to the subject a therapeutically effective amount of the present polynucleotide(s) or composition.

At least one type of vector may be administered by injection into an eye of the subject.

The Cas protein or enzyme/nuclease may be a wildtype (wt) Cas (e.g., a wildtype (wt) Cas9), a Cas nickase (e.g., a Cas9 nickase), or a dCas (e.g., a dCas9).

In one embodiment, the dCas is fused to a repressor domain, such as a Krüppel-associated Box (KRAB) domain, or any other repressor domain as described herein including combinations thereof.

In another embodiment, the guide RNA comprises at least one PUF (Pumilio mRNA binding factor) binding sequence, which may bind to, e.g., PUF or the PUF-KRAB fusion protein.

The autosomal dominant disease-related gene may be BEST1, RHO, PRDM13, RGR, TEAD1, AIPL1, CRX, GUCA1A, GUCY2D, PITPNM3, PROM1, PRPH2, RIMS1, SEMA4A, UNC119, GNAT1, PDE6B, WSF1, IMPDH1, OTX2, C1QTNF5, CTNNA1, EFEMP1, ELOVL4, FSCN2, GUCA1B, HMCN1, IMPG1, RP1L1, TIMP3, VCAN, MFN2, NR2F1, OPA1, ARL3, CA4, HK1, KLHL7, NR2E3, NRL, PRPF3, PRPF4, PRPF6, PRPF8, PRPF31, RDH12, ROM1, RP1, RP9, RPE65, SNRNP200, SPP2, TOPORS, ABCC6, ATXN7, COL11A1, COL2A1, JAG1, KCNJ13, KIF11, OPA3, PAX2, TREX1, CAPN5, CRB1, FZD4, ITM2B, LRP5, MAPKAPK3, MIR204, OPN1SW, RB1, TSPAN12, or ZNF408.

In certain embodiments, the autosomal dominant disease-related gene is BEST1 or RHO.

The mutations of the disease-related gene may be single base mutations, missense mutations (including single missense mutations), deletions, etc.

The mutations of the disease-related gene may be in one or more coding regions, in one or more non-coding regions, in one or more intergenic regions (regions between genes), or in combinations of one or more coding regions, and/or one or more non-coding regions, and/or intergenic regions. The mutations may be in one or more introns, in one or more exons, or in a combination of one or more introns and one or more exons.

The polynucleotide/vector may be a recombinant adeno-associated viral (AAV) vector, such as an AAV2 vector, or an AAV8 vector.

The present method and system may treat or prevent a dominantly-inherited condition (such as an ocular disease) in a subject, or modify a gene in a cell from a subject (or derived from a cell from a subject) having a dominantly-inherited condition (such as an ocular disease).

The ocular diseases include, but are not limited to, vitelliform macular dystrophy (VMD), Best vitelliform macular dystrophy, autosomal dominant chorioretinal atrophy or degeneration, autosomal dominant cone or cone-rod dystrophy, autosomal dominant congenital stationary night blindness, autosomal dominant Leber congenital amaurosis, autosomal dominant macular degeneration, autosomal dominant ocular-retinal developmental disease, autosomal dominant optic atrophy, (autosomal dominant) retinitis pigmentosa, autosomal dominant syndromic/systemic diseases with retinopathy, sorsby macular dystrophy, age-related macular degeneration, doyne honeycomb macular disease, and juvenile macular degeneration.

In one embodiment, the ocular disease is age-related macular degeneration. In another embodiment, the ocular disease is juvenile macular degeneration.

The present system may be delivered by any suitable means. In certain embodiments, the system is delivered in vivo. In other embodiments, the system is delivered to isolated/cultured cells (e.g., autologous iPS cells) in vitro to provide modified cells useful for in vivo delivery to a subject/patient.

As an alternative to injection of viral particles described in the present disclosure, cell replacement therapy can be used to prevent, correct or treat diseases, where the methods of the present disclosure are applied to isolated patient's cells (ex vivo), which is then followed by the injection of “corrected” cells back into the patient.

In one embodiment, the disclosure provides for introducing one or more vectors as described herein into a eukaryotic cell. The cell may be a stem cell. Examples of stem cells include pluripotent, multipotent and unipotent stem cells. Examples of pluripotent stem cells include embryonic stem cells, embryonic germ cells, embryonic carcinoma cells and induced pluripotent stem cells (iPSCs).

For the treatment of ocular diseases, patient's iPS cells can be isolated and differentiated into retinal pigment epithelium (RPE) cells ex vivo. Patient's iPS cells or RPE cells characterized by the mutation in autosomal dominant disease-related gene may be manipulated using methods of the present disclosure in a manner that results in the ablation (e.g., deletion) or silencing (e.g., transcription blocked) of a disease-related gene.

Thus, the present disclosure provides methods for correcting autosomal dominant ocular disease in a subject, wherein the method results in the ablation of a disease-related gene. The method may comprise administering to the subject a therapeutically effective amount of autologous or allogeneic retinal pigment RPE cells with the ablated disease-related gene. Administration of the pharmaceutical preparations comprising RPE cells with the ablated disease-related gene may be effective to reduce the severity of symptoms and/or to prevent further deterioration in the patient's condition. Such administration may be effective to fully restore any vision loss or other symptoms.

“Induced pluripotent stem cells,” commonly abbreviated as iPS cells or iPSCs, refer to a type of pluripotent stem cell artificially prepared from a non-pluripotent cell, typically an adult somatic cell, or terminally differentiated cell, such as a fibroblast, a hematopoietic cell, a myocyte, a neuron, an epidermal cell, or the like, by introducing certain factors, referred to as reprogramming factors.

The present methods may further comprise differentiating the iPS cell to a differentiated cell, for example, an ocular cell.

For example, patient fibroblast cells can be collected from the skin biopsy and transformed into iPS cells. Dimos J T et al. (2008) Induced pluripotent stem cells generated from patients with ALS can be differentiated into motor neurons. Science 321: 1218-1221; Nature Reviews Neurology 4, 582-583 (November 2008). Luo et al., Generation of induced pluripotent stem cells from skin fibroblasts of a patient with olivopontocerebellar atrophy, Tohoku J. Exp. Med. 2012, 226(2): 151-9. The CRISPR-mediated modification can be done at this stage. The corrected cell clone can be screened and selected by RFLP assay. The corrected cell clone is then differentiated into RPE cells and tested for its RPE-specific markers (e.g., Bestrophinl, RPE65, Cellular Retinaldehyde-binding Protein, and MFRP). Well-differentiated RPE cells can be transplanted autologously back to the donor patient.

The cell may be autologous or allogeneic to the subject who is administered the cell.

The term “autologous” refers to any material derived from the same individual to whom it is later to be re-introduced into the same individual.

The term “allogeneic” refers to any material derived from a different animal of the same species as the individual to whom the material is introduced. Two or more individuals of the same species are said to be allogeneic to one another.

The corrected cells for cell therapy to be administered to a subject. Cells (e.g., RPE cells) described in the present disclosure may be formulated with a pharmaceutically acceptable carrier. For example, cells can be administered alone or as a component of a pharmaceutical formulation. The cells (e.g., RPE cells) can be administered in combination with one or more pharmaceutically acceptable sterile isotonic aqueous or nonaqueous solutions (e.g., balanced salt solution (BSS)), dispersions, suspensions or emulsions, or sterile powders which may be reconstituted into sterile injectable solutions or dispersions just prior to use, which may contain antioxidants, buffers, bacteriostats, solutes or suspending or thickening agents.

The present system may be delivered into the retina of a subject. The present system may be administered through injections, such as subretinal or intravitreal injections.

The corrected cells (e.g., RPE cells) may be delivered in a pharmaceutically acceptable ophthalmic formulation by intraocular injection. Concentrations for injections may be at any amount that is effective and nontoxic. The pharmaceutical preparations of the cells of the present disclosure for treatment of a patient may be formulated at doses of at least about 10⁴ cells/mL. The cell preparations for treatment of a patient can be formulated at doses of at least or about 10³, 10⁴, 10⁵, 10⁶, 10⁷, 10⁸, 10⁹, or 10¹⁰ cells/mL.

Subjects, which may be treated according to the present disclosure, include all animals which may benefit from the present invention. Such subjects include mammals, preferably humans (infants, children, adolescents and/or adults), but can also be an animal such as dogs and cats, farm animals such as cows, pigs, sheep, horses, goats and the like, and laboratory animals (e.g., rats, mice, guinea pigs, and the like).

The term “nuclease” is used to generally refer to any enzyme that hydrolyzes nucleic acid sequences.

The term “ocular cells” refers to any cell in, or associated with the function of, the eye. The term may refer to any one or more of photoreceptor cells, including rod, cone and photosensitive ganglion cells, retinal pigment epithelium (RPE) cells, Mueller cells, bipolar cells, horizontal cells, or amacrine cells. In one embodiment, the ocular cells are bipolar cells. In another embodiment, the ocular cells are horizontal cells. In yet a third embodiment, the ocular cells include ganglion cells.

The terms “polynucleotide”, “nucleotide”, “nucleotide sequence”, “nucleic acid” and “oligonucleotide” are used interchangeably. These terms refer to a polymeric form of nucleotides of any length, either deoxyribonucleotides or ribonucleotides, or analogs. Examples of polynucleotides include, but are not limited to, coding or non-coding regions of a gene or gene fragment, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. One or more nucleotides within a polynucleotide sequence can further be modified. The sequence of nucleotides may be interrupted by non-nucleotide components. A polynucleotide may also be modified after polymerization, such as by conjugation with a labeling agent.

Bestrophin-1 (Best1) is a protein that is encoded by the BEST1 gene, in humans (RPD ID—5T5N/4RDQ). The bestrophin family of proteins comprises four evolutionary related genes (BEST1, BEST2, BEST3, and BEST4) that code for integral membrane proteins. This gene family is characterized by proteins with a highly conserved N-terminus with four to six transmembrane domains. Specifically, the BEST1 gene on chromosome 11q12.3 encodes the Bestrophin-1 protein in humans whose expression is highest in the retina. Bestrophins may form chloride ion channels or may regulate voltage-gated L-type calcium-ion channels. Bestrophins are generally believed to form calcium-activated chloride-ion channels in epithelial cells but they have also been shown to be highly permeable to bicarbonate ion transport in retinal tissue. Mutations in this gene are responsible for juvenile-onset vitelliform macular dystrophy (VMD2), also known as Best macular dystrophy, in addition to adult-onset vitelliform macular dystrophy (AVMD) and other retinopathies. Alternative splicing results in multiple variants encoding distinct isoforms. In humans, BEST1 mutations have been linked with Best vitelliform macular dystrophy (BVMD). Mutations in the BEST1 gene have been identified as the primary cause for at least five different degenerative retinal diseases.

The NCBI Reference Sequence (RefSeq) accession numbers for human BEST1 mRNA may include NM_001139443, NM_001300786, NM_001300787, NM_004183 and NM_001363591. The NCBI Reference Sequence (RefSeq) accession numbers for human BEST1 protein may include NP_001132915, NP_001287715, NP_001287716, NP_004174, and NP_001350520. The NCBI Reference Sequence (RefSeq) accession numbers for murine BEST1 mRNA may include NM_011913. The NCBI Reference Sequence (RefSeq) accession numbers for murine BEST1 protein may include NP_036043.

The mutants of BEST1 include, but are not limited to, pR218H, pL234P, and pA243T.

As used herein, the term “CRISPR” refers to a technique of sequence specific genetic manipulation relying on the clustered regularly interspaced short palindromic repeats pathway. CRISPR can be used to perform gene editing and/or gene regulation, as well as to simply target proteins to a specific genomic location. Gene editing refers to a type of genetic engineering in which the nucleotide sequence of a target polynucleotide is changed through introduction of deletions, insertions, or base substitutions to the polynucleotide sequence. In some aspects, CRISPR-mediated gene editing utilizes the pathways of non-homologous end-joining (NHEJ) or homologous recombination to perform the edits. Gene regulation refers to increasing or decreasing the production of specific gene products such as protein or RNA.

The term “Cas9” refers to a CRISPR associated endonuclease referred to by this name. Non-limiting exemplary Cas9s are provided herein, e.g. the Cas9 provided for in UniProtKB G3ECR1 (CAS9_STRTR) or the Staphylococcus aureus Cas9, as well as the nuclease dead Cas9, orthologs and biological equivalents each thereof. Orthologs include but are not limited to Streptococcus pyogenes Cas9 (“spCas9”); Cas 9 from Streptococcus thermophiles, Legionella pneumophilia, Neisseria lactamica, Neisseria meningitides, Francisella novicida; and Cpf1 (which performs cutting functions analogous to Cas9) from various bacterial species including Acidaminococcus spp. and Francisella novicida U112.

The term “gRNA” or “guide RNA” as used herein refers to the guide RNA sequences used to target specific genes for correction employing the CRISPR technique. Techniques of designing gRNAs and donor therapeutic polynucleotides for target specificity are well known in the art. For example, Doench, J., et al. Nature biotechnology 2014; 32(12):1262-7, Mohr, S. et al. (2016) FEBS Journal 283: 3232-38, and Graham, D., et al. Genome Biol. 2015; 16: 260. gRNA may comprise, or alternatively consist essentially of, or yet further consist of, a fusion polynucleotide comprising CRISPR RNA (crRNA) and trans-activating CRIPSPR RNA (tracrRNA); or a polynucleotide comprising CRISPR RNA (crRNA) and trans-activating CRIPSPR RNA (tracrRNA). In some aspects, a gRNA is synthetic (Kelley, M. et al. (2016) J of Biotechnology 233 (2016) 74-83). As used herein, a biological equivalent of a gRNA includes but is not limited to polynucleotides or targeting molecules that can guide a Cas or equivalent thereof to a specific nucleotide sequence such as a specific region of a cell's genome.

The Cas protein or enzyme/nuclease may be a wildtype (wt) Cas (e.g., a wildtype (wt) Cas9), a Cas nickase (e.g., a Cas9 nickase), or a dCas (e.g., a dCas9).

A nuclease-defective or nuclease-deficient Cas protein (e.g., dCas9) with one or more mutations on its nuclease domains retains DNA binding activity when complexed with gRNA. dCas protein can tether and localize effector domains or protein tags by means of protein fusions to sites matched by gRNA, thus constituting an RNA-guided DNA binding enzyme. dCas can be fused to transcriptional activation domain (e.g., VP64) or repressor domain (e.g., KRAB), and be guided by gRNA to activate or repress target genes, respectively. dCas can also be fused with fluorescent proteins and achieve live-cell fluorescent labeling of chromosomal regions.

gRNAs can be generated to target a specific gene, optionally a gene associated with a disease, disorder, or condition. Thus, in combination with Cas, the guide RNAs facilitate the target specificity of the CRISPR/Cas system. Further aspects such as promoter choice, as discussed herein, may provide additional mechanisms of achieving target specificity—e.g., selecting a promoter for the guide RNA encoding polynucleotide that facilitates expression in a particular organ or tissue. Accordingly, the selection of suitable gRNAs for the particular disease, disorder, or condition is contemplated herein.

In some embodiments, the nucleotide sequence encoding the Cas (e.g., Cas9) nuclease is modified to alter the activity of the protein. In some embodiments, the Cas (e.g., Cas9) nuclease is a catalytically inactive Cas (e.g., Cas9) (or a catalytically deactivated/defective Cas9 or dCas9). In one embodiment, dCas (e.g., dCas9) is a Cas protein (e.g., Cas9) that lacks endonuclease activity due to point mutations at one or both endonuclease catalytic sites (RuvC and HNH) of wild type Cas (e.g., Cas9). For example, dCas9 contains mutations of catalytically active residues (D10 and H840) and does not have nuclease activity. In some cases, the dCas has a reduced ability to cleave both the complementary and the non-complementary strands of the target DNA. As a non-limiting example, in some cases, the dCas9 harbors both D10A and H840A mutations of the amino acid sequence of S. pyogenes Cas9. In some embodiments when a dCas9 has reduced or defective catalytic activity (e.g., when a Cas9 protein has a D10, G12, G17, E762, H840, N854, N863, H982, H983, A984, D986, and/or a A987 mutation, e.g., D10A, G12A, G17A, E762A, H840A, N854A, N863A, H982A, H983A, A984A, and/or D986A), the Cas protein can still bind to target DNA in a site-specific manner, because it is still guided to a target polynucleotide sequence by a DNA-targeting sequence of the subject polynucleotide (e.g., gRNA), as long as it retains the ability to interact with the Cas-binding sequence of the subject polynucleotide (e.g., gRNA).

Inactivation of Cas endonuclease activity can create a catalytically deactivated Cas (dCas, e.g., dCas9). dCas can bind but not cleave DNA, thus preventing the transcription of the target gene by creating a physical barrier to the action of transcription factors. This rendition of CRISPR works at the transcription level in a reversible fashion. This strategy has been termed CRISPR interference, or CRISPRi. In CRISPR interference (CRISPRi), dCas fusion proteins (e.g., dCas fused to another protein or portion thereof) may be used in the present method for gene repression. In some embodiments, dCas is fused to a (transcriptional) repressor domain or a transcriptional silencer. Non-limiting examples of transcriptional repression domains include a Krüppel-associated Box (KRAB) domain, an ERF repressor domain (ERD), a mSin3A interaction domain (SID) domain, concatemers of SID (e.g. SID4X), or a homolog thereof. Non-limiting examples of transcriptional silencers include Heterochromatin Protein 1 (HP1). CRISPRi may be modified by fusing Cas (e.g., dCas) to the Kruppel-associated box repression domain (KRAB), which augments the repressive effects of Cas. Gilbert et al. CRISPR-mediated modular RNA-guided regulation of transcription in eukaryotes. Cell. 2013; 154(2):442-51.

Second generation CRISPRi strongly represses via PUF-KRAB repressors. PUF proteins (named after Drosophila Pumilio and C. elegans fern-3 binding factor) are known to be involved in mediating mRNA stability and translation. These proteins contain a unique RNA-binding domain known as the PUF domain. The RNA-binding PUF domain, such as that of the human Pumilio 1 protein (referred here also as PUM), contains 8 repeats (each repeat called a PUF motif or a PUF repeat) that bind consecutive bases in an anti-parallel fashion, with each repeat recognizing a single base, i.e., PUF repeats R1 to R8 recognize nucleotides N8 to N1, respectively. For example, PUM is composed of eight tandem repeats, each repeat consisting of 34 amino acids that folds into tightly packed domains composed of alpha helices. PUF and its derivatives or functional variants are programmable RNA-binding domains that can be used in the present methods and systems, as part of a PUF domain-fusion that brings any effector domain to a specific PUF-binding sequence on the subject polynucleotide (e.g., gRNA).

As used herein, “PUF domain” refers to a wildtype or naturally existing PUF domain, as well as a PUF homologue domain that is based on/derived from a natural or existing PUF domain, such as the prototype human Pumilio 1 PUF domain.

The PUF adaptor protein binds gRNA modified with a PUF-binding sequence. The gRNA may comprise one or more PUF-binding sequences. For example, the gRNA can be derived by inserting multiple copies of short PUF-binding sequences (e.g., 8-mer), e.g., downstream of gRNA stem loops or upstream of the target-matching region. In certain embodiments, each of the one or more copies of the PUF-binding sequence has about 8 nucleotides. The gRNA may have more than one copies of the PUF-binding sequence. In certain embodiments, the gRNA comprises about 1, 2, 3, 4, 5, 10, 15, 20, 25, 30, 35, 40, 45, 46, 47, 48, 49, 50, 5-15 copies, about 5-14 copies, about 5-13 copies, about 5-12 copies, about 5-11 copies, about 5-10 copies, or about 5-9 copies of the PUF-binding sequence, such as 5, 6, 7, 8, 9, 10, 11, 12, 13, 14, or 15 copies of PUF-binding sequence. In certain embodiments, the range of the PUF-binding sequence copy number is L to H, wherein L is any one of 1, 2, 3, 4, 5, 6, 7, 8, 9, 10, 11, 12, 13, 14, 15, 20, 25, 30, 35, or 40, and wherein H is any one of 2, 3, 4, 5, 6, 7, 8, 9, 10, 11, 12, 13, 14, 15, 20, 25, 30, 35, 40, 45, 50, 55, 60, 65, 70, 80, 90, or 100, so long as H is greater than L. Each PUF-binding sequence may be the same or different. The PUF-binding sequence may be those disclosed in U.S. Patent Publication No. 20180094257, the content of which is incorporated herein by reference in its entirety. The gRNA may comprise one or more tandem sequences, each of which can be specifically recognized and bound by a specific PUF domain. Since a PUF domain can be engineered to bind virtually any PUF-binding sequence based on the nucleotide-specific interaction between the individual PUF motifs of PUF domain and the single RNA nucleotide they recognize, the PUF-binding sequence sequences can be any designed sequence that bind their corresponding PUF domain. In certain embodiments, a PUF-binding sequence of the invention has 8-mer. In other embodiments, a PUF-binding sequence has 5, 6, 7, 8, 9, 10, 11, 12, 13, 14, 15, 16 or more RNA nucleotides. The PUF-binding sequence may bind the human Pumilio 1 PUF domain (wildtype or mutant).

In certain embodiments, the PUF domain comprises PUF motifs from different PUF domains from different proteins. For example, a PUF domain may be constructed with PUF motifs from the human Pumilio 1 protein and one or more other PUF motifs from one or more other PUF proteins, such as PuDp or FBF. In certain embodiments, the PUF domain is a Pumilio homology domain (PU-HUD). In a particular embodiment, the PU-HUD is a human Pumilio 1 domain. In certain embodiments, the PUF domain is any PUF protein family member with a Pum-HD domain. Non-limiting examples of a PUF family member include FBF in C. elegans, Ds pum in Drosophila, and PUF proteins in plants such as Arabidopsis and rice. Tam et al., The Puf family of RNA-binding proteins in plants: phylogeny, structural modeling, activity and subcellular localization, BMC Plant Biol. 10:44, 2010, the entire contents of which are incorporated by reference herein.

The present methods and systems may use CRISPR deletion (CRISPRd). CRISPRd capitalizes on the tendency of DNA repair strategies to default towards NHEJ, and does not require a donor template to repair the cleaved strand. Instead, Cas creates a DSB in the gene harboring a mutation first, then NHEJ occurs, and insertions and/or deletions (INDELs) are introduced that corrupt the sequence, thus either preventing the gene from being expressed or proper protein folding from occurring. This strategy may be particularly applicable for dominant conditions, in which case knocking out the mutated, dominant allele and leaving the wild type allele intact may be sufficient to restore the phenotype to wild type.

In certain embodiments, the Cas enzyme may be a catalytically defective Cas (e.g., Cas9) or dCas, or a Cas nickase or nickase.

The Cas enzyme (e.g., Cas9) may be modified to function as a nickase, named as such because it “nicks” the DNA by inducing single-strand breaks instead of DSBs. The term “Cas nickase” or “nickase”, as used herein, refers to a Cas protein that is capable of cleaving only one strand of a duplexed nucleic acid molecule (e.g., a duplexed DNA molecule). In some embodiments, a Cas nickase may be any of the nickase disclosed in U.S. Pat. No. 10,167,457, the content of which is incorporated herein by reference in its entirety. In one embodiment, a Cas (e.g., Cas9) nickase has an active HNH nuclease domain and is able to cleave the non-targeted strand of DNA, i.e., the strand bound by the gRNA. In one embodiment, a Cas (e.g., Cas9) nickase has an inactive RuvC nuclease domain and is not able to cleave the targeted strand of the DNA, i.e., the strand where base editing is desired. In some embodiments the Cas nickase cleaves the target strand of a duplexed nucleic acid molecule, meaning that the Cas nickase cleaves the strand that is base paired to (complementary to) a gRNA (e.g., an sgRNA) that is bound to the Cas. In some embodiments, the Cas nickase cleaves the non-target, non-base-edited strand of a duplexed nucleic acid molecule, meaning that the Cas nickase cleaves the strand that is not base paired to a gRNA (e.g., an sgRNA) that is bound to the Cas. In one embodiment, the Cas9 nickase is a Cas9 D10A nickase bearing a mutation in the RuvC endonuclease domain. Additional suitable Cas9 nickases will be apparent to those of skill in the art based on this disclosure and knowledge in the field, and are within the scope of this disclosure.

In an embodiment, the composition comprises HNH-like domain cleavage activity but having no, or no significant, N-terminal RuvC-like domain cleavage activity. In an embodiment, the composition comprises N-terminal RuvC-like domain cleavage activity but having no, or no significant, HNH-like domain cleavage activity.

For example, the nickase can be a Cas9 nickase with a mutation at a position corresponding to D10A of S. pyogenes Cas9; or the nickase can be a Cas9 nickase with a mutation at a position corresponding to H840A of S. pyogenes Cas9. In some cases, the Cas nickase can cleave the complementary strand of the target DNA but has reduced ability to cleave the non-complementary strand of the target DNA. For example, the Cas9 nickase can have a mutation (e.g., an amino acid substitution) that reduces the function of the RuvC domain. As a non-limiting example, the Cas9 nickase is a D10A mutation of the amino acid sequence of S. pyogenes Cas9. In some cases, the Cas9 nickase can cleave the non-complementary strand of the target DNA but has reduced ability to cleave the complementary strand of the target DNA. For example, the Cas9 nickase can have a mutation (e.g., an amino acid substitution) that reduces the function of the HNH domain (RuvC/HNH/RuvC domain motifs). As a non-limiting example, the Cas9 nickase is a H840A of S. pyogenes Cas9. U.S. Patent Publication No. 20180094257, the content of which is incorporated herein by reference in its entirety.

In one embodiment, two gRNAs targeting sites that are close together can direct separate Cas nickases to induce breaks on each DNA strand. By requiring two different cleavage events to induce recombination, this strategy can decrease the likelihood of off-targeting effects.

In CRISPR activation (CRISPRa), dCas may be fused to an activator domain, such as VP64 or VPR. Such dCas fusion proteins may be used with the constructs described herein for gene activation. In some embodiments, dCas is fused to an epigenetic modulating domain, such as a histone demethylase domain or a histone acetyltransferase domain. In some embodiments, dCas is fused to a LSD1 or p300, or a portion thereof. In some embodiments, the dCas fusion is used for CRISPR-based epigenetic modulation. In some embodiments, dCas or Cas is fused to a Fok1 nuclease domain. In some embodiments, Cas or dCas fused to a Fok1 nuclease domain is used for genome editing. In some embodiments, Cas or dCas is fused to a fluorescent protein (e.g., GFP, RFP, mCherry, etc.). In some embodiments, Cas/dCas proteins fused to fluorescent proteins are used for labeling and/or visualization of genomic loci or identifying cells expressing the Cas endonuclease.

The present disclosure provides for gene editing methods that can modify the disease-related gene, which in turn can be used for in vivo gene therapy for patients afflicted with the disease.

The present disclosure takes advantage of the CRISPR gene-editing system. In certain embodiments, the method uses a gene-editing enzyme with one or multiple unique guide RNA (gRNA, such as single guide RNA or sgRNA) sequences. This targeting may or may not be followed by supplying the wild type gene cDNA, that may or may not be codon modified in order to evade recognition, by the sgRNA(s).

Cas9 harbors two independent nuclease domains homologous to HNH and RuvC endonucleases, and by mutating either of the two domains, the Cas9 protein can be converted to a nickase that introduces single-strand breaks (Cong, L. et al. Science 339, 819-823 (2013)). It is specifically contemplated that the methods and compositions of the present disclosure can be used with the single- or double-strand-inducing version of Cas9, as well as with other RNA-guided DNA nucleases, such as other bacterial Cas9-like systems.

The sequence-specific nuclease of the present methods and compositions described herein can be engineered, chimeric, or isolated from an organism. The nuclease can be introduced into the cell in the form of a DNA, mRNA and protein.

In one embodiment, the DNA digesting agent can be a site-specific nuclease. In another embodiment, the site-specific nuclease may be a Cas-family nuclease. In a more specific embodiment, the Cas nuclease may be a Cas9 nuclease.

In one embodiment, Cas protein may be a functional derivative of a naturally occurring Cas protein.

In addition to well characterized CRISPR-Cas system, a new CRISPR enzyme, called Cpf1 (Cas protein 1 of PreFran subtype) may be used in the present methods and systems (Zetsche et al. Cell. pii: S0092-8674(15)01200-3. doi: 10.1016/j.cell.2015.09.038 (2015)). Cpf1 is a single RNA-guided endonuclease that lacks tracrRNA, and utilizes a T-rich protospacer-adjacent motif. The authors demonstrated that Cpf1 mediates strong DNA interference with characteristics distinct from those of Cas9. Thus, in one embodiment of the present invention, CRISPR-Cpf1 system can be used to cleave a desired region within the targeted gene.

Any suitable nuclease may be used in the present methods and systems. Nucleases are enzymes that hydrolyze nucleic acids. Nucleases may be classified as endonucleases or exonucleases. An endonuclease is any of a group of enzymes that catalyze the hydrolysis of bonds between nucleic acids in the interior of a DNA or RNA molecule. An exonuclease is any of a group of enzymes that catalyze the hydrolysis of single nucleotides from the end of a DNA or RNA chain. Nucleases may also be classified based on whether they specifically digest DNA or RNA. A nuclease that specifically catalyzes the hydrolysis of DNA may be referred to as a deoxyribonuclease or DNase, whereas a nuclease that specifically catalyzes the hydrolysis of RNA may be referred to as a ribonuclease or an RNase. Some nucleases are specific to either single-stranded or double-stranded nucleic acid sequences. Some enzymes have both exonuclease and endonuclease properties. In addition, some enzymes are able to digest both DNA and RNA sequences.

Non-limiting examples of the endonucleases include a zinc finger nuclease (ZFN), a ZFN dimer, a ZFNickase, a transcription activator-like effector nuclease (TALEN), or a RNA-guided DNA endonuclease (e.g., CRISPR/Cas). Meganucleases are endonucleases characterized by their capacity to recognize and cut large DNA sequences (12 base pairs or greater). Any suitable meganuclease may be used in the present methods to create double-strand breaks in the host genome, including endonucleases in the LAGLIDADG and PI-Sce family.

In further embodiment, the nuclease is a transcription activator-like effector nuclease (TALEN). TALENs contains a TAL effector domain that binds to a specific nucleotide sequence and an endonuclease domain that catalyzes a double strand break at the target site (PCT Patent Publication No. WO2011072246; Miller et al., Nat. Biotechnol. 29, 143-148 (2011); Cermak et al., Nucleic Acid Res. 39, e82 (2011)). Sequence-specific endonucleases may be modular in nature, and DNA binding specificity is obtained by arranging one or more modules. Bibikova et al., Mol. Cell. Biol. 21, 289-297 (2001). Boch et al., Science 326, 1509-1512 (2009).

ZFNs can contain two or more (e.g., 2-8, 3-6, 6-8, or more) sequence-specific DNA binding domains (e.g., zinc finger domains) fused to an effector endonuclease domain (e.g., the FokI endonuclease). Porteus et al., Nat. Biotechnol. 23, 967-973 (2005). Kim et al. (2007) Hybrid restriction enzymes: Zinc finger fusions to Fok I cleavage domain, Proceedings of the National Academy of Sciences of USA, 93: 1156-1160. U.S. Pat. No. 6,824,978. PCT Publication Nos. WO1995/09233 and WO1994018313.

In one embodiment, the nuclease is a site-specific nuclease of the group or selected from the group consisting of omega, zinc finger, TALE, and CRISPR/Cas.

The sequence-specific endonuclease of the methods and compositions described here can be engineered, chimeric, or isolated from an organism. Endonucleases can be engineered to recognize a specific DNA sequence, by, e.g., mutagenesis. Seligman et al. (2002) Mutations altering the cleavage specificity of a homing endonuclease, Nucleic Acids Research 30: 3870-3879. Combinatorial assembly is a method where protein subunits form different enzymes can be associated or fused. Arnould et al. (2006) Engineering of large numbers of highly specific homing endonucleases that induce recombination to novel DNA targets, Journal of Molecular Biology 355: 443-458. In certain embodiments, these two approaches, mutagenesis and combinatorial assembly, can be combined to produce an engineered endonuclease with desired DNA recognition sequence.

The sequence-specific nuclease can be introduced into the cell in the form of a protein or in the form of a nucleic acid encoding the sequence-specific nuclease, such as an mRNA or a cDNA. Nucleic acids can be delivered as part of a larger construct, such as a plasmid or viral vector, or directly, e.g., by electroporation, lipid vesicles, viral transporters, microinjection, and biolistics. Similarly, the construct containing the one or more transgenes can be delivered by any method appropriate for introducing nucleic acids into a cell.

Single guide RNA(s) used in the methods of the present disclosure can be designed so that they direct binding of the Cas-sgRNA complexes to pre-determined cleavage sites in a genome. In one embodiment, the cleavage sites may be chosen so as to release a fragment or sequence that contains a region of autosomal dominant disease-related gene. In further embodiment, the cleavage sites may be chosen so as to release a fragment or sequence that contains a region of an autosomal dominant disease-related gene.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule, which can form hydrogen bonds (e.g., Watson-Crick base pairing) with a second nucleic acid sequence. Full complementarity is not necessarily required, provided there is sufficient complementarity to cause hybridization and promote formation of a CRISPR complex. A target sequence may comprise any polynucleotide, such as DNA or RNA polynucleotides. The Cas9 protein can tolerate mismatches distal from the PAM. The PAM sequence varies by the species of the bacteria from which Cas9 was derived. The most widely used CRISPR system is derived from S. pyogenes and the PAM sequence is NGG located on the immediate 3′ end of the sgRNA recognition sequence. The PAM sequences of CRISPR systems from exemplary bacterial species include: Streptococcus pyogenes (NGG), Neisseria meningitidis (NNNNGATT), Streptococcus thermophilus (NNAGAA) and Treponema denticola (NAAAAC).

sgRNA(s) used in the present disclosure can be between about 5 and 100 nucleotides long, or longer (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91 92, 93, 94, 95, 96, 97, 98, 99, or 100 nucleotides in length, or longer). In one embodiment, sgRNA(s) can be between about 15 and about 30 nucleotides in length (e.g., about 15-29, 15-26, 15-25; 16-30, 16-29, 16-26, 16-25; or about 18-30, 18-29, 18-26, or 18-25 nucleotides in length).

To facilitate sgRNA design, many computational tools have been developed (See Prykhozhij et al. (PLoS ONE, 10(3): (2015)); Zhu et al. (PLoS ONE, 9(9) (2014)); Xiao et al. (Bioinformatics. Jan. 21 (2014)); Heigwer et al. (Nat Methods, 11(2): 122-123 (2014)). Methods and tools for guide RNA design are discussed by Zhu (Frontiers in Biology, 10 (4) pp 289-296 (2015)), which is incorporated by reference herein.

The present methods and systems may be used to prevent, correct, or treat ocular diseases that arise due to the presence of autosomal dominant mutation. Non-limiting examples of ocular diseases include vitelliform macular dystrophy (VMD), such as Best vitelliform macular dystrophy (BVMD) or juvenile-onset vitelliform macular dystrophy, and adult-onset vitelliform macular dystrophy (AVMD); autosomal recessive bestrophinopathy; autosomal dominant vitreoretinochoroidopathy and retinitis pigmentosa (RP). Non-limiting examples of ocular diseases include retinopathies, retinal dystrophies, and retinal degenerative diseases.

The present systems and methods can be used as a gene-editing tool for the correction of the mutation(s) found in any autosomal dominant disease. Thus, the methods of the present disclosure can be used to treat any autosomal dominant disease, including, but not limited to, Acropectoral syndrome, Acute intermittent porphyria, Adermatoglyphia, Albright's hereditary osteodystrophy, Arakawa's syndrome II, Aromatase excess syndrome, Autosomal dominant cerebellar ataxia, Autosomal dominant retinitis pigmentosa, Axenfeld syndrome, Bethlem myopathy, Birt-Hogg-Dubé syndrome, Boomerang dysplasia, Branchio-oto-renal syndrome, Buschke-Ollendorff syndrome, Camurati-Engelmann disease, Central core disease, Collagen disease, Collagenopathy, types II and XI, Congenital distal spinal muscular atrophy, Congenital stromal corneal dystrophy, Costello syndrome, Currarino syndrome, Darier's disease, De Vivo disease, Dentatorubral-pallidoluysian atrophy, Dermatopathia pigmentosa reticularis, DiGeorge syndrome, Doyne honeycomb disease, Dysfibrinogenemia, Familial amyloid polyneuropathy, Familial atrial fibrillation, Familial hypercholesterolemia, Familial male-limited precocious puberty, Feingold syndrome, Felty's syndrome, Flynn-Aird syndrome, Gardner's syndrome, Gillespie syndrome, Gray platelet syndrome, Greig cephalopolysyndactyly syndrome, Hajdu-Cheney syndrome, Hawkinsinuria, Hay-Wells syndrome, Hereditary elliptocytosis, Hereditary hemorrhagic telangiectasia, Hereditary mucoepithelial dysplasia, Hereditary spherocytosis, Holt-Oram syndrome, Huntington's disease, Hypertrophic cardiomyopathy, Hypoalphalipoproteinemia, Hypochondroplasia, Jackson-Weiss syndrome, Keratolytic winter erythema, Kniest dysplasia, Kostmann syndrome, Langer-Giedion syndrome, Larsen syndrome, Liddle's syndrome, Marfan syndrome, Marshall syndrome, Medullary cystic kidney disease, Metachondromatosis, Miller-Dieker syndrome, MOMO syndrome, Monilethrix, Multiple endocrine neoplasia, Multiple endocrine neoplasia type 1, Multiple endocrine neoplasia type 2, Multiple endocrine neoplasia type 2b, Myelokathexis, Myotonic dystrophy, Naegeli-Franceschetti-Jadassohn syndrome, Nail-patella syndrome, Noonan syndrome, Oculopharyngeal muscular dystrophy, Pachyonychia congenital, Pallister-Hall syndrome, PAPA syndrome, Papillorenal syndrome, Parastremmatic dwarfism, Pelger-Huet anomaly, Peutz-Jeghers syndrome, Polydactyly, Popliteal pterygium syndrome, Porphyria cutanea tarda, Pseudoachondroplasia, RASopathy, Reis-Bucklers corneal dystrophy, Romano-Ward syndrome, Rosselli-Gulienetti syndrome, Roussy-Lévy syndrome, Rubinsteinn-Taybi syndrome, Saethre-Chotzen syndrome, Schmitt Gillenwater Kelly syndrome, Short QT syndrome, Singleton Merten syndrome, Spinal muscular atrophy with lower extremity predominance, Spinocerebellar ataxia, Spinocerebellar ataxia type 6, Spondyloepiphyseal dysplasia congenital, Spondyloperipheral dysplasia, Stickler syndrome, Tietz syndrome, Timothy syndrome, Treacher Collins syndrome, Tuberous sclerosis, Upington disease, Variegate porphyria, Vitelliform macular dystrophy, Von Hippel-Lindau disease, Von Willebrand disease, Wallis-Zieff-Goldblatt syndrome, WHIM syndrome, White sponge nevus, Worth syndrome, Zaspopathy, Zimmermann-Laband syndrome, and Zori-Stalker-Williams syndrome. The present systems and methods can be used as a gene-editing tool to prevent, correct, or treat autosomal dominant kidney diseases such as renal angiomyolipomas, medullary cystic kidney disease, or autosomal dominant polycystic kidney disease.

Examples of such ocular diseases also include, but are not limited, autosomal dominant chorioretinal atrophy or degeneration, autosomal dominant cone or cone-rod dystrophy, autosomal dominant congenital stationary night blindness, autosomal dominant Leber congenital amaurosis, autosomal dominant macular degeneration, autosomal dominant ocular-retinal developmental disease, autosomal dominant optic atrophy, autosomal dominant retinitis pigmentosa, autosomal dominant syndromic/systemic diseases with retinopathy, sorsby macular dystrophy, age-related macular degeneration, doyne honeycomb macular disease, and juvenile macular degeneration.

The methods of the present disclosure can be used for arresting progression of or ameliorating vision loss associated with retinitis pigmentosa (RP) in the subject.

Vision loss may include decrease in peripheral vision, central (reading) vision, night vision, day vision, loss of color perception, loss of contrast sensitivity, or reduction in visual acuity. The methods of the present disclosure can also be used to prevent, or arrest photoreceptor function loss, or increase photoreceptor function in the subject.

RP is diagnosed in part, through an examination of the retina. The eye exam usually reveals abnormal, dark pigment deposits that streak the retina. Additional tests for diagnosing RP include electroretinogram (ERG) and visual field testing.

Methods for measuring or assessing visual function, retinal function (such as responsiveness to light stimulation), or retinal structure in a subject are well known to one of skill in the art. See, e.g. Kanski's Clinical Ophthalmology: A Systematic Approach, Edition 8, Elsevier Health Sciences, 2015. Methods for measuring or assessing retinal response to light include may include detecting an electrical response of the retina to a light stimulus. This response can be detected by measuring an electroretinogram (ERG; for example, full-field ERG, multifocal ERG, or ERG photostress test), visual evoked potential, or optokinetic nystagmus (see, e.g., Wester et al., Invest. Ophthalmol. Vis. Sci. 48:4542-4548, 2007). Furthermore, retinal response to light may be measured by directly detecting retinal response (for example by use of a microelectrode at the retinal surface). ERG has been extensively described by Vincent et al. Retina, 2013; 33(1):5-12. Thus, methods of the present disclosure can be used to improve visual function, retinal function (such as responsiveness to light stimulation), retinal structure, or any other clinical symptoms or phenotypic changes associated with ocular diseases in subjects afflicted with ocular disease.

In one embodiment, the methods of the present disclosure can be used to prevent the development and progression of autosomal dominant disease. For example, a patient may be a carrier of autosomal dominant mutation, but the phenotypic expression of a disease has not been yet manifested, although the genomic defect has been identified by screening. The methods of the present disclosure may be applied to such patient to prevent the onset of disease.

In addition to being used for the prevention, correctness, or treatment of autosomal dominant diseases, the methods of the present disclosure can be used to prevent, correct, or treat any autosomal recessive diseases. Thus, all the methods described here as applicable to autosomal dominant diseases and autosomal dominant genes or fragments can be adopted for use in the treatment of autosomal recessive diseases.

In further embodiments, the methods of the present disclosure can be used to prevent, correct, or treat ocular diseases that arise due to the presence of autosomal recessive mutation. Examples of such diseases include, but are not limited to, autosomal recessive congenital stationary night, autosomal recessive deafness alone or syndromic, autosomal recessive Leber congenital amaurosis, autosomal recessive optic atrophy, autosomal recessive retinitis pigmentosa, autosomal recessive syndromic/systemic diseases with retinopathy, autosomal recessive usher syndrome, other autosomal recessive retinopathy, autosomal recessive cone or cone-rod dystrophy, autosomal recessive macular degeneration, and autosomal recessive bardet-biedl syndrome.

Mutations in various genes have been identified to give rise to autosomal dominant diseases (such genes are also referred to as autosomal dominant disease-related genes). The methods of the present disclosure can be used to fully or partially correct mutations in such autosomal dominant disease-related genes.

In all cases where accession numbers are used, the accession numbers refer to one embodiment of the gene which may be used with the methods of the present disclosure. In one embodiment, the accession numbers are NCBI (National Center for Biotechnology Information) reference sequence (RefSeq) numbers.

In certain embodiments, the disease-related gene is an autosomal dominant disease-related gene, or an autosomal recessive disease-related gene.

For example, the autosomal dominant disease-related gene in retinitis pigmentosa may include, but are not limited to, ARL3(NC_000010.11 (102673727 . . . 102714433, complement)), BEST1 (e.g., NG_009033.1), CA4 (NG_012050.1), CRX (NG_008605.1), FSCN2 (NG_015964.1), GUCA1B (NG_016216.1), HK1 (NG_012077.1), IMPDH1 (NG_009194.1), KLHL7 (NG_016983.1), NR2E3 (NG_009113.2), NRL (NG_011697.1), PRPF3 (NG_008245.1), PRPF4 (NG_034225.1), PRPF6 (NG_029719.1), PRPF8 (NG_009118.1), PRPF31 (NG_009759.1), PRPH2 (NG_009176.1), RDH12 (NG_008321.1), RHO (NG_009115.1), ROM1 (NG_009845.1), RP1 (NG_009840.1), RP9 (NG_012968.1), RPE65 (NG_008472.1), SEMA4A (NG_027683.1), SNRNP200 (NG_016973.1), SPP2 (NG_008668.1), and TOPORS (NG_017050.1). Genes and mutations causing autosomal dominant retinitis pigmentosa are in detail discussed by Daiger et al. (Cold Spring Harb Perspect Med. 2014 Oct. 10; 5(10)).

Another type of the autosomal dominant disease-related gene is autosomal dominant chorioretinal atrophy or degeneration-related gene, which may include: PRDM13 (NC_000006.12 (99606774 . . . 99615578)), RGR (NG_009106.1), and TEAD1 (NG_021302.1).

Another example of the autosomal dominant disease-related gene is autosomal dominant cone or cone-rod dystrophy-related gene, which can include: AIPL1 (NG_008474.1), CRX (NG_008605.1), GUCA1A (NG_009938.1), GUCY2D (NG_009092.1), PITPNM3 (NG_016020.1), PROM1 (NG_011696.1), PRPH2 (NG_009176.1), RIMS1 (NG_016209.1), SEMA4A (NG_027683.1), and UNC119 (NG_012302.1).

In one embodiment, the autosomal dominant disease-related gene is autosomal dominant congenital stationary night blindness-related gene, including: GNAT1 (NG_009831.1), PDE6B (NG_009839.1), and RHO (NG_009115.1).

In another embodiment, the autosomal dominant disease-related gene is autosomal dominant deafness (alone or syndromic)-related gene such as WSF1(NC_000004.12 (6269850 . . . 6303265)).

Another type of the autosomal dominant disease-related gene is autosomal dominant Leber congenital amaurosis-related gene, which may include: CRX(NG_008605.1), (NG_009194.1), and OTX2(NG_008204.1).

Another example of the autosomal dominant disease-related gene is autosomal dominant macular degeneration-related gene, which can include: BEST1(NG_009033.1), C1QTNF5 (NG_012235.1), CTNNA1 (NC_000005.10 (138753396 . . . 138935034)), EFEMP1 (NG_009098.1), ELOVL4 (NG_009108.1), FSCN2 (NG_015964.1), GUCA1B (NG_016216.1), HMCN1 (NG_011841.1), IMPG1 (NG_041812.1), OTX2 (NG_008204.1), PRDM13 (NC_000006.12 (99606774 . . . 99615578)), PROM1 (NG_011696.1), PRPH2 (NG_009176.1), RP1L1 (NG_028035.1), and TIMP3(NG_009117.1).

In one embodiment, the autosomal dominant disease-related gene is autosomal dominant ocular retinal developmental disease-related gene such as VCAN(NG_012682.1). The accession numbers are provided as specific examples of each gene which may be used with the methods of the disclosure.

In another embodiment, the autosomal dominant disease-related gene is autosomal dominant optic atrophy-related gene, including: MFN2 (NG_007945.1), NR2F1 (NG_034119.1), and OPA1 (NG_011605.1).

In one embodiment, the autosomal dominant disease-related gene is autosomal dominant syndromic/systemic disease with retinopathy-related gene, including: ABCC6 (NG_007558.2), ATXN7 (NG_008227.1), COL11A1 (NG_008033.1), COL2A1 (NG_008072.1), JAG1 (NG_007496.1), KCNJ13 (NG_016742.1), KIF11 (NG_032580.1), MFN2 (NG_007945.1), OPA3 (NG_013332.1), PAX2 (NG_008680.2), TREX1 (NG_009820.1), and VCAN (NG_012682.1).

Another example of the autosomal dominant disease-related gene is autosomal dominant retinopathy-related gene, including: BEST1 (NG_009033.1), CAPN5 (NG_033002.1), CRB1 (NG_008483.2), FZD4 (NG_011752.1), ITM2B (NG_013069.1), LRP5 (NG_015835.1), MAPKAPK3 (NC_000003.12(50611862 . . . 50649297)), MIR204 (NR_029621.1), OPN1SW (NG_009094.1), RB1 (NG_009009.1), TSPAN12 (NG_023203.1), and ZNF408 (NC_000011.10 (46700767 . . . 46705916).

According to the methods described here, autosomal recessive disease-related gene is corrected and can in-part or fully restore the function of a wild-type gene.

One type of the autosomal recessive disease-related gene is congenital stationary night-related gene, including: CABP4(NG_021211.1), GNAT1(NG_009831.1), GNB3 (NG_009100.1), GPR179(NG_032655.2), GRK1(NC_000013.11(113667279 . . . 113671659)), GR M6(NG_008105.1), LRIT3(NG_033249.1), RDH5(NG_008606.1), SAG(NG_009116.1), SLC24 A1(NG_031968.2), and TRPM1(NG_016453.2).

Another type of the autosomal recessive disease-related gene is bardet-biedl syndrome-related gene, including: ADIPOR1 (NC_000001.1 (202940825 . . . 202958572, complement)), ARL6 (NG_008119.2), BBIP1 (NG_041778.1), BBS1 (NG_009093.1), BBS2 (NG_009312.1), BBS4 (NG_009416.2), BBS5 (NG_011567.1), BBS7 (NG_009111.1), BBS9 (NG_009306.1), BBS10 (NG_016357.1), BBS12 (NG_021203.1), C8orf37 (NG_032804.1), CEP290 (NG_008417.1), IFT172 (NG_034068.1), IFT27 (NG_034205.1), INPP5E (NG_016126.1), KCNJ13 (NG_016742.1), LZTFL1 (NG_033917.1), MKKS (NG_009109.1), MKS1 (NG_013032.1), NPHP1 (NG_008287.1), SDCCAG8 (NG_027811.1), TRIM32 (NG_011619.1), and TTC8 (NG_008126.1).

One example of the autosomal recessive disease-related gene is cone or cone-rod dystrophy-related gene, including, but not limited to, ABCA4(NG_009073.1), ADAMS (NG_016335.1), ATF6 (NG_029773.1), C21orf2 (NG_032952.1), C8orf37 (NG_032804.1), CACNA2D4 (NG_012663.1), CDHR1 (NG_028034.1), CERKL (NG_021178.1), CNGA3 (NG_009097.1), CNGB3 (NG_016980.1), CNNM4 (NG_016608.1), GNAT2 (NG_009099.1), KCNV2 (NG_012181.1), PDE6C (NG_016752.1), PDE6H (NG_016859.1), POC1B (NG_041783.1), RAB28 (NG_033891.1), RAX2 (NG_011565.1), RDH5 (NG_008606.1), RPGRIP1 (NG_008933.1), and TTLL5(NG_016974.1).

Another example of the autosomal recessive disease-related gene is deafness (alone or syndromic)-related gene including: CDH23(NG_008835.1), CIB2(NG_033006.1), DFNB31 (NG_016700.1), MYO7A (NG_009086.1), PCDH15 (NG_009191.2), PDZD7 (NG_028030.1), and USH1C(NG_011883.1).

In one embodiment, the autosomal recessive disease-related gene is Leber congenital amaurosis-related gene, including: AIPL1(NG_008474.1), CABP4(NG_021211.1), CEP290 (NG_008417.1), CLUAP1 (NC_000016.10(3500945 . . . 3539048)), CRB1 (NG_008483.2), CRX (NG_008605.1), DTHD1 (NG_032962.1), GDF6 (NG_008981.1), GUCY2D (NG_009092.1), IFT140 (NG_032783.1), IQCB1 (NG_015887.1), KCNJ13 (NG_016742.1), LCA5 (NG_016011.1), LRAT (NG_009110.1), NMNAT1 (NG_032954.1), PRPH2 (NG_009176.1), RD3 (NG_013042.1), RDH12 (NG_008321.1), RPE65 (NG_008472.1), RPGRIP1 (NG_008933.1), SPATA7 (NG_021183.1), and TULP1 (NG_009077.1).

In another embodiment, the autosomal recessive disease-related gene is optic atrophy-related gene, including: RTN4IP1(NC_000006.12 (106571028 . . . 106630500, complement)), SLC25A46 (NC_000005.10 (110738136 . . . 110765161)), and TMEM126A(NG_017157.1).

One example of the autosomal recessive disease-related gene is retinitis pigmentosa-related gene, including: ABCA4 (NG_009073.1), AGBL5 (NC_000002.12 (27051423 . . . 27070622)), ARL6 (NG_008119.2), ARL2BP (NG_033905.1), BBS1 (NG_009093.1), BBS2 (NG_009312.1), BEST1 (NG_009033.1), C2orf71 (NG_021427.1), C8orf37 (NG_032804.1), CERKL (NG_021178.1), CLRN1 (NG_009168.1), CNGA1 (NG_009193.1), CNGB1 (NG_016351.1), CRB1 (NG_008483.2), CYP4V2 (NG_007965.1), DHDDS (NG_029786.1), DHX38 (NG_034207.1), EMC1 (NG_032948.1), EYS (NG_023443.2), FAM161A (NG_028125.1), GPR125 (NC_000004.12 (22387374 . . . 22516058, complement)), HGSNAT(NG_009552.1), IDH3B (NG_012149.1), IFT140 (NG_032783.1), IFT172 (NG_034068.1), IMPG2 (NG_028284.1), KIAA1549 (NG_032965.1), KIZ (NG_033122.1), LRAT (NG_009110.1), MAK (NG_030040.1), MERTK (NG_011607.1), MVK (NG_007702.1), NEK2 (NG_029112.1), NEUROD1 (NG_011820.1), NR2E3 (NG_009113.2), NRL (NG_011697.1), PDE6A (NG_009102.1), PDE6B (NG_009839.1), PDE6G (NG_009834.1), POMGNT1 (NG_009205.2), PRCD (NG_016702.1), PROM1 (NG_011696.1), RBP3(NG_029718.1), RGR(NG_009106.1), RHO(NG_009115.1), RLBP1(NG_008116.1), RP1(NG_009840.1), RP1L1(NG_028035.1), RPE65(NG_008472.1), SAG(NG_009116.1), SLC7A14(NG_034121.1), SPATA7(NG_021183.1), TTC8(NG_008126.1), TULP1(NG_009077 0.1), USH2A(NG_009497.1), ZNF408(NC_000011.10 (46700767 . . . 46705916)), and ZNF513 (NG_028219.1).

Another example of the autosomal recessive disease-related gene is syndromic/systemic disease with retinopathy-related gene, including: ABCC6(NG_007558.2), ABHD12 (NG_028119.1), ACBD5(NG_032960.2), ADAMTS18(NG_031879.1), ADIPOR1(NC_000001. 11(202940825 . . . 202958572, complement)), AHI1(NG_008643.1), ALMS1(NG_011690.1), CC2D2A(NG_013035.1), CEP164(NG_033032.1), CEP290(NG_008417.1), CLN3(NG_008654 0.2), COL9A1(NG_011654.1), CSPP1(NG_034100.1), ELOVL4(NG_009108.1), EXOSC2 (NC_000009.12 (130693760 . . . 130704894)), FLVCR1(NG_028131.1), FLVCR1 (NG_028131.1), GNPTG(NG_016985.1), HARS(NG_032158.1), HGSNAT(NG_009552.1), H MX1(NG_013062.2), IFT140(NG_032783.1), INPP5E(NG_016126.1), INVS(NG_008316.1), IQ CB1(NG_015887.1), LAMA1(NG_034251.1), LRP5(NG_015835.1), MKS1(NG_013032.1), M TTP(NG_011469.1), NPHP1(NG_008287.1), NPHP3(NG_008130.1), NPHP4(NG_011724.2), 0 PA3(NG_013332.1), PANK2(NG_008131.3), PCYT1A(NG_042817.1), PEX1(NG_008341.1), PEX2(NG_008371.1), PEX7(NG_008462.1), PHYH(NG_012862.1), PLK4(NG_041821.1), PNP LA6(NG_013374.1), POC1B(NG_041783.1), PRPS1(NG_008407.1), RDH11(NG_042282.1), RPGRIP1L(NG_008991.2), SDCCAG8(NG_027811.1), SLC25A46(NC_000005.10(110738136. 0.110765161)), TMEM237(NG_032049.1), TRNT1(NG_041800.1), TTPA(NG_016123.1), TUB(NG_029912.1), TUBGCP4(NG_042168.1), TUBGCP6(NG_032160.1), WDPCP(NG_028144.1), WDR19(NG_031813.1), WFS1(NG_011700.1), and ZNF423(NG_032972.2).

One type of the autosomal recessive disease-related gene is usher syndrome-related gene, including: ABHD12(NG_028119.1), CDH23(NG_008835.1), CEP250 (NC_000020.11 (35455139 . . . 35517531)), CIB2(NG_033006.1), CLRN1(NG_009168.1), DFNB31(NG_016700.1), GPR98(NG_007083.1), HARS(NG_032158.1), MYO7A(NG_009086.1), PCDH15(NG_00919 1.2), USH1C(NG_011883.1), USH1G(NG_007882.1), and USH2A(NG_009497.1).

Another type of the autosomal recessive disease-related gene is retinopathy-related gene, including: BEST1(NG_009033.1), C12orf65(NG_027517.1), CDH3(NG_009096.1), CNGA3 (NG_009097.1), CNGB3(NG_016980.1), CNNM4(NG_016608.1), CYP4V2(NG_007965.1), LRP5(NG_015835.1), MFRP(NG_012235.1), MVK(NG_007702.1), NBAS (NG_032964.1), NR2E3(NG_009113.2), OAT(NG_008861.1), PLA2G5(NG_032045.1), PROM1(NG_011696.1), RBP4(NG_009104.1), RGS9(NG_013021.1), RGS9BP(NG_016751.1), and RLBP1 (NG_008116.1).

Yet another type of the autosomal recessive disease-related gene is macular degeneration-related gene, including: ABCA4(NG_009073.1), CFH(NG_007259.1), DRAM2 (NC_000001.11 (111117332 . . . 111140216, complement)), IMPG1(NG_041812.1), and MFSD8(NG_008657.1).

In addition to being used for the prevention, correctness, or treatment of autosomal dominant and recessive diseases, the methods of the present disclosure can be used to prevent, correct, or treat any X-linked diseases. Thus, all the methods described here as applicable to autosomal dominant diseases and autosomal dominant genes or fragments can be adopted for use in the treatment of X-linked diseases.

Furthermore, the methods of the present disclosure can be used to prevent, correct, or treat ocular diseases that arise due to the presence of X-linked mutation. Examples of such diseases include: X-linked cone or cone-rod dystrophy, X-linked congenital stationary night blindness, X-linked macular degeneration, X-linked retinitis pigmentosa, X-linked syndromic/systemic diseases with retinopathy, X-linked optic atrophy, and X-linked retinopathies. According to the methods described here, X-linked disease-related gene is corrected and can in part or fully restore the function of a wild-type gene.

One example of the X-linked disease-related gene is cone or cone-rod dystrophy-related gene, including: CACNA1F (NG_009095.2) and RPGR (NG_009553.1).

Another example of the X-linked disease-related gene is congenital stationary night blindness-related gene, including: CACNA1F (NG_009095.2) and NYX (NG_009112.1).

In one embodiment, the X-linked disease-related gene is macular degeneration-related gene, such as RPGR (NG_009553.1).

In another embodiment, the X-linked disease-related gene is optic atrophy-related gene, such as TIMM8A (NG_011734.1).

One type of the X-linked disease-related gene is retinitis pigmentosa-related gene, including: OFD1 (NG_008872.1), RP2 (NG_009107.1), and RPGR (NG_009553.1).

Another type of the X-linked disease-related gene is syndromic/systemic disease with retinopathy-related gene, including: OFD1(NG_008872.1) and TIMM8A(NG_011734.1).

Yet another example of the X-linked disease-related gene is retinopathy-related gene, including, CACNA1F (NG_009095.2), CHM (NG_009874.2), DMD (NG_012232.1), NDP (NG_009832.1), OPN1LW(NG_009105.2), OPN1MW(NG_011606.1), PGK1(NG_008862.1), and RS1(NG_008659.3).

The disease-related gene may be HTRA1.

In another embodiment, the methods of the present disclosure can be used to prevent, correct, or treat diseases that arise due to the presence of mutation in mitochondrial DNA. Such diseases may include, retinopathy caused by the gene mutations in mitochondrial DNA. Examples of genes that may be characterized by the mutation in mitochondrial DNA that causes the development of retinopathy include: MT-ATP6(NC_012920.1 (8527 . . . 9207)), MT-TH(NC_012920.1 (12138 . . . 12206)), MT-TL1(NC_012920.1 (3230 . . . 3304)), MT-TP(NC_012920.1 (15956 . . . 16023, complement), and MT-TS2(NC_012920.1 (12207 . . . 12265)). Table 1 provides an exemplary list of diseases and disease-related genes (accompanied with corresponding accession numbers) that can be treated and/or corrected using methods of the present disclosure.

TABLE 1
Disease Related Disorders and Genes

| Disease Category | Mapped and Identified Genes |
|---|---|
| Bardet-Biedl | ADIPOR1(NC_000001.11 (202940825 . . . 202958572, |
| syndrome, autosomal | complement)), ARL6(NG_008119.2), BBIP1(NG_041778.1), |
| recessive | BBS1(NG_009093.1), BBS2(NG_009312.1), BBS4(NG_009416.2), |
| | BBS5(NG_011567.1), BBS7(NG_009111.1), BBS9(NG_009306.1), |
| | BBS10(NG_016357.1), BBS12(NG_021203.1), C8orf37(NG_032804.1), |
| | CEP290(NG_008417.1), IFT172(NG_034068.1), IFT27(NG_034205.1), |
| | INPP5E(NG_016126.1), KCNJ13(NG_016742.1), LZTFL1(NG_033917.1), |
| | MKKS(NG_009109.1), MKS1(NG_013032.1), NPHP1(NG_008287.1), |
| | SDCCAG8(NG_027811.1), TRIM32(NG_011619.1), TTC8(NG_008126.1) |
| Chorioretinal atrophy | PRDM13(NC_000006.12 |
| or degeneration, | (99606774 . . . 99615578)), RGR(NG_009106.1), TEAD1(NG_021302.1) |
| autosomal dominant | |
| Cone or cone-rod | AIPL1(NG_008474.1), CRX(NG_008605.1), GUCA1A(NG_009938.1), |
| dystrophy, autosomal | GUCY2D(NG_009092.1), PITPNM3(NG_016020.1), |
| dominant | PROM1(NG_011696.1), PRPH2(NG_009176.1), RIMS1(NG_016209.1), |
| | SEMA4A(NG_027683.1), UNC119(NG_012302.1) |
| Cone or cone-rod | ABCA4(NG_009073.1), ADAM9(NG_016335.1), ATF6(NG_029773.1), |
| dystrophy, autosomal | C21orf2(NG_032952.1), C8orf37(NG_032804.1), CACNA2D4(NG_012663.1), |
| recessive | CDHR1(NG_028034.1), CERKL(NG_021178.1), CNGA3(NG_009097.1), |
| | CNGB3(NG_016980.1), CNNM4(NG_016608.1), GNAT2(NG_009099.1), |
| | KCNV2(NG_012181.1), PDE6C(NG_016752.1), PDE6H(NG_016859.1), |
| | POC1B(NG_041783.1), RAB28(NG_033891.1), RAX2(NG_011565.1), |
| | RDH5(NG_008606.1), RPGRIP1(NG_008933.1), TTLL5(NG_016974.1) |
| Cone or cone-rod | CACNA1F(NG_009095.2), RPGR(NG_009553.1) |
| dystrophy, X-linked | |
| Congenital stationary | GNAT1(NG_009831.1), PDE6B(NG_009839.1), RHO(NG_009115.1) |
| night blindness, | |
| autosomal dominant | |
| Congenital stationary | CABP4(NG_021211.1), GNAT1(NG_009831.1), GNB3(NG_009100.1), |
| night blindness, | GPR179(NG_032655.2), GRK1(NC_000013.11 |
| autosomal recessive | (113667279 . . . 113671659)), GRM6(NG_008105.1), LRIT3(NG_033249.1), |
| | RDH5(NG_008606.1), SAG(NG_009116.1), SLC24A1(NG_031968.2), |
| | TRPM1(NG_016453.2) |
| Congenital stationary | CACNA1F(NG_009095.2), NYX(NG_009112.1) |
| night blindness, X- | |
| linked | |
| Deafness alone or | WSF1(NC_000004.12 (6269850 . . . 6303265)) |
| syndromic, | |
| autosomal dominant | |
| Deafness alone or | CDH23(NG_008835.1), CIB2(NG_033006.1), DFNB31(NG_016700.1), |
| syndromic, | MYO7A(NG_009086.1), PCDH15(NG_009191.2), PDZD7(NG_028030.1), |
| autosomal recessive | USH1C(NG_011883.1) |
| Leber congenital | CRX(NG_008605.1), IMPDH1(NG_009194.1), OTX2(NG_008204.1) |
| amaurosis, autosomal | |
| dominant | |
| Leber congenital | AIPL1(NG_008474.1), CABP4(NG_021211.1), CEP290(NG_008417.1), |
| amaurosis, autosomal | CLUAP1(NC_000016.10 |
| recessive | (3500945 . . . 3539048)), CRB1(NG_008483.2), CRX(NG_008605.1), |
| | DTHD1(NG_032962.1), GDF6(NG_008981.1), GUCY2D(NG_009092.1), |
| | IFT140(NG_032783.1), IQCB1(NG_015887.1), KCNJ13(NG_016742.1), |
| | LCA5(NG_016011.1), LRAT(NG_009110.1), NMNAT1(NG_032954.1), |
| | PRPH2(NG_009176.1), RD3(NG_013042.1), RDH12(NG_008321.1), |
| | RPE65(NG_008472.1), RPGRIP1(NG_008933.1), |
| | SPATA7(NG_021183.1), TULP1(NG_009077.1) |
| Macular | BEST1(NG_009033.1), C1QTNF5(NG_012235.1), |
| degeneration, | CTNNA1(NC_000005.10 |
| autosomal dominant | (138753396 . . . 138935034)), EFEMP1(NG_009098.1), |
| | ELOVL4(NG_009108.1), FSCN2(NG_015964.1), GUCA1B(NG_016216.1), |
| | HMCN1(NG_011841.1), IMPG1(NG_041812.1), OTX2(NG_008204.1), |
| | PRDM13(NC_000006.12 (99606774 . . . 99615578)), |
| | PROM1(NG_011696.1), PRPH2(NG_009176.1), |
| | RP1L1(NG_028035.1), TIMP3(NG_009117.1) |
| Macular | ABCA4(NG_009073.1), CFH(NG_007259.1), DRAM2(NC_000001.11 |
| degeneration, | (111117332 . . . 111140216, |
| autosomal recessive | complement)), IMPG1(NG_041812.1), MFSD8(NG_008657.1) |
| Macular | RPGR(NG_009553.1) |
| degeneration, X- | |
| linked | |
| Ocular-retinal | VCAN(NG_012682.1) |
| developmental | |
| disease, autosomal | |
| dominant | |
| Optic atrophy, | MFN2(NG_007945.1), NR2F1(NG_034119.1), OPA1(NG_011605.1) |
| autosomal dominant | |
| Optic atrophy, | RTN4IP1(NC_000006.12 (106571028 . . . 106630500, |
| autosomal recessive | complement)), SLC25A46(NC_000005.10 |
| | (110738136 . . . 110765161)), TMEM126A(NG_017157.1) |
| Optic atrophy, X- | TIMM8A(NG_011734.1) |
| linked | |
| Retinitis pigmentosa, | ARL3(NC_000010.11 (102673727 . . . 102714433, |
| autosomal dominant | complement)), BEST1(NG_009033.1), CA4(NG_012050.1), |
| | CRX(NG_008605.1), FSCN2(NG_015964.1), GUCA1B(NG_016216.1), |
| | HK1(NG_012077.1), IMPDH1(NG_009194.1), KLHL7(NG_016983.1), |
| | NR2E3(NG_009113.2), NRL(NG_011697.1), PRPF3(NG_008245.1), |
| | PRPF4(NG_034225.1), PRPF6(NG_029719.1), PRPF8(NG_009118.1), |
| | PRPF31(NG_009759.1), PRPH2(NG_009176.1), RDH12(NG_008321.1), |
| | RHO(NG_009115.1), ROM1(NG_009845.1), RP1(NG_009840.1), |
| | RP9(NG_012968.1), RPE65(NG_008472.1), SEMA4A(NG_027683.1), |
| | SNRNP200(NG_016973.1), SPP2(NG_008668.1), TOPORS(NG_017050.1) |
| Retinitis pigmentosa, | ABCA4(NG_009073.1), AGBL5(NC_000002.12 |
| autosomal recessive | (27051423 . . . 27070622)), ARL6(NG_008119.2), ARL2BP(NG_033905.1), |
| | BBS1(NG_009093.1), BBS2(NG_009312.1), BEST1(NG_009033.1), |
| | C2orf71(NG_021427.1),C8orf37(NG_032804.1), CERKL(NG_021178.1), |
| | CLRN1(NG_009168.1), CNGA1(NG_009193.1), CNGB1(NG_016351.1), |
| | CRB1(NG_008483.2), CYP4V2(NG_007965.1), DHDDS(NG_029786.1), |
| | DHX38(NG_034207.1), EMC1(NG_032948.1), EYS(NG_023443.2), |
| | FAM161A(NG_028125.1), GPR125(NC_000004.12 |
| | (22387374 . . . 22516058, |
| | complement)), HGSNAT(NG_009552.1), IDH3B(NG_012149.1), |
| | IFT140(NG_032783.1), IFT172(NG_034068.1), IMPG2(NG_028284.1), |
| | KIAA1549(NG_032965.1), KIZ(NG_033122.1), LRAT(NG_009110.1), |
| | MAK(NG_030040.1), MERTK(NG_011607.1), MVK(NG_007702.1), |
| | NEK2(NG_029112.1), NEUROD1(NG_011820.1), NR2E3(NG_009113.2), |
| | NRL(NG_011697.1), PDE6A(NG_009102.1), PDE6B(NG_009839.1), |
| | PDE6G(NG_009834.1), POMGNT1(NG_009205.2), PRCD(NG_016702.1), |
| | PROM1(NG_011696.1), RBP3(NG_029718.1), RGR(NG_009106.1), |
| | RHO(NG_009115.1), RLBP1(NG_008116.1), RP1(NG_009840.1), |
| | RP1L1(NG_028035.1), RPE65(NG_008472.1), SAG(NG_009116.1), |
| | SLC7A14(NG_034121.1), SPATA7(NG_021183.1), TTC8(NG_008126.1), |
| | TULP1(NG_009077.1), USH2A(NG_009497.1), ZNF408(NC_000011.10 |
| | (46700767 . . . 46705916)), ZNF513(NG_028219.1) |
| Retinitis pigmentosa, | OFD1(NG_008872.1), RP2(NG_009107.1, RPGR(NG_009553.1) |
| X-linked | |
| Syndromic/systemic | ABCC6(NG_007558.2), ATXN7(NG_008227.1), COL11A1(NG_008033.1), |
| diseases with | COL2A1(NG_008072.1), JAG1(NG_007496.1), KCNJ13(NG_016742.1), |
| retinopathy, | KIF11(NG_032580.1), MFN2(NG_007945.1), OPA3(NG_013332.1), |
| autosomal dominant | PAX2(NG_008680.2), TREX1(NG_009820.1), VCAN(NG_012682.1) |
| Syndromic/systemic | ABCC6(NG_007558.2), ABHD12(NG_028119.1), ACBD5(NG_032960.2), |
| diseases with | ADAMTS18(NG_031879.1), ADIPOR1(NC_000001.11 |
| retinopathy, | (202940825 . . . 202958572, complement)), |
| autosomal recessive | AHI1(NG_008643.1), ALMS1(NG_011690.1), CC2D2A(NG_013035.1), |
| | CEP164(NG_033032.1), CEP290(NG_008417.1), CLN3(NG_008654.2), |
| | COL9A1(NG_011654.1), CSPP1(NG_034100.1), |
| | ELOVL4(NG_009108.1), EXOSC2(NC_000009.12 |
| | (130693760 . . . 130704894)), FLVCR1(NG_028131.1), |
| | GNPTG(NG_016985.1), HARS(NG_032158.1), HGSNAT(NG_009552.1), |
| | HMX1(NG_013062.2), IFT140(NG_032783.1), INPP5E(NG_016126.1), |
| | INVS(NG_008316.1), IQCB1(NG_015887.1), LAMA1(NG_034251.1), |
| | LRP5(NG_015835.1), MKS1(NG_013032.1), MTTP(NG_011469.1), |
| | NPHP1(NG_008287.1), NPHP3(NG_008130.1), |
| | NPHP4(NG_011724.2), OPA3(NG_013332.1), PANK2(NG_008131.3), |
| | PCYT1A(NG_042817.1), PEX1(NG_008341.1), PEX2(NG_008371.1), |
| | PEX7(NG_008462.1), PHYH(NG_012862.1), PLK4(NG_041821.1), |
| | PNPLA6(NG_013374.1), POC1B(NG_041783.1), PRPS1(NG_008407.1), |
| | RDH11(NG_042282.1), RPGRIP1L(NG_008991.2), SDCCAG8(NG_027811.1), |
| | SLC25A46(NC_000005.10 (110738136 . . . 110765161)), |
| | TMEM237(NG_032049.1), TRNT1(NG_041800.1), TTPA(NG_016123.1), |
| | TUB(NG_029912.1), TUBGCP4(NG_042168.1), TUBGCP6(NG_032160.1), |
| | WDPCP(NG_028144.1), WDR19(NG_031813.1), WFS1(NG_011700.1), |
| | ZNF423(NG_032972.2) |
| Syndromic/systemic | OFD1(NG_008872.1), TIMM8A(NG_011734.1) |
| diseases with | |
| retinopathy, X-linked | |
| Usher syndrome, | ABHD12(NG_028119.1), CDH23(NG_008835.1), CEP250(NC_000020.11 |
| autosomal recessive | (35455139 . . . 35517531)), CIB2(NG_033006.1), |
| | CLRN1(NG_009168.1), DFNB31(NG_016700.1), GPR98(NG_007083.1), |
| | HARS(NG_032158.1), MYO7A(NG_009086.1), PCDH15(NG_009191.2), |
| | USH1C(NG_011883.1), USH1G(NG_007882.1), USH2A(NG_009497.1) |
| Other retinopathy, | BEST1(NG_009033.1), CAPN5(NG_033002.1), CRB1(NG_008483.2), |
| autosomal dominant | FZD4(NG_011752.1), ITM2B(NG_013069.1), LRP5(NG_015835.1), |
| | MAPKAPK3(NC_000003.12 (50611862 . . . 50649297)), |
| | MIR204(NR_029621.1), OPN1SW(NG_009094.1), RB1(NG_009009.1), |
| | TSPAN12(NG_023203.1), ZNF408(NC_000011.10 |
| | (46700767 . . . 46705916)) |
| Other retinopathy, | BEST1(NG_009033.1), C12orf65(NG_027517.1), CDH3(NG_009096.1), |
| autosomal recessive | CNGA3(NG_009097.1), CNGB3(NG_016980.1), CNNM4(NG_016608.1), |
| | CYP4V2(NG_007965.1), LRP5(NG_015835.1), MFRP(NG_012235.1), |
| | MVK(NG_007702.1), NBAS(NG_032964.1), NR2E3(NG_009113.2), |
| | OAT(NG_008861.1), PLA2G5(NG_032045.1), PROM1(NG_011696.1), |
| | RBP4(NG_009104.1), RGS9(NG_013021.1), RGS9BP(NG_016751.1), |
| | RLBP1(NG_008116.1) |
| Other retinopathy, | MT-ATP6(NC_012920.1 (8527 . . . 9207)), MT-TH(NC_012920.1 |
| mitochondrial | (12138 . . . 12206)), MT-TL1(NC_012920.1 (3230 . . . 3304)), |
| | MT-TP(NC_012920.1 (15956 . . . 16023, complement)), |
| | MT-TS2(NC_012920.1 (12207 . . . 12265)) |
| Other retinopathy, X- | CACNA1F(NG_009095.2), CHM(NG_009874.2), DMD(NG_012232.1), |
| linked | NDP(NG_009832.1), OPN1LW(NG_009105.2), |
| | OPN1MW(NG_011606.1), PGK1(NG_008862.1), RS1(NG_008659.3) |

The methods of the present disclosure can also be used to prevent, correct, or treat cancers that arise due to the presence of mutation in a tumor suppressor gene. Examples of tumor suppression genes include: retinoblastoma susceptibility gene (RB) gene, p53 gene, deleted in colon carcinoma (DCC) gene, adenomatous polyposis coli (APC) gene, p16, BRCA1, BRCA2, MSH2, and the neurofibromatosis type 1 (NF-1) tumor suppressor gene (Lee at al. Cold Spring Harb Perspect Biol. 2010 October; 2(10)).

Tumor suppressor genes are genes that, in their wild-type alleles, express proteins that suppress abnormal cellular proliferation. When the gene coding for a tumor suppressor protein is mutated or deleted, the resulting mutant protein or the complete lack of tumor suppressor protein expression may fail to correctly regulate cellular proliferation, and abnormal cellular proliferation may take place, particularly if there is already existing damage to the cellular regulatory mechanism. A number of well-studied human tumors and tumor cell lines have been shown to have missing or nonfunctional tumor suppressor genes. Thus, a loss of function or inactivation of tumor suppressor genes may play a central role in the initiation and/or progression of a significant number of human cancers.

The methods of the present disclosure may be used to treat patients at a different stage of the disease (e.g. early, middle or late). The present methods may be used to treat a patient once or multiple times. Thus, the length of treatment may vary and may include multiple treatments. As discussed in the present disclosure, the methods or the present disclosure can be used for correcting or treating autosomal dominant ocular disease in a subject.

A variety of viral constructs may be used to deliver the present system to the targeted cells and/or a subject. Non-limiting examples of such recombinant viruses include recombinant adeno-associated virus (AAV), recombinant adenoviruses, recombinant lentiviruses, recombinant retroviruses, recombinant poxviruses, and other known viruses in the art, as well as plasmids, cosmids, and phages. Options for gene delivery viral constructs are well known (see, e.g., Ausubel et al., Current Protocols in Molecular Biology, John Wiley & Sons, New York, 1989; Kay, M. A., et al., 2001 Nat. Medic. 7(1):33-40; and Walther W. and Stein U., 2000 Drugs, 60(2): 249-71).

The present system can be delivered to the subject or cell using one or more recombinant adeno-associated viral (AAV) vectors (e.g., 1, 2, 3, 4, 5, 6, 7, 8, 9, or more AAV vectors). One or more gRNAs (e.g., sgRNAs) can be packaged into a single (one) recombinant AAV vector or more vectors. A Cas-family nuclease can be packaged into the same, or alternatively separate recombinant AAV vectors. Wirth et al. Gene. 2013 Aug. 10; 525(2):162-9.

The method described here also provides for correcting autosomal dominant ocular disease in a subject, comprising administering to said subject by injection a therapeutically effective amount of a recombinant AAV virus encoding the present system.

As the carrying capacity of AAV may pose challenges, two or more AAV vectors can be used simultaneously. For example, a Cas family nuclease may be packaged into a different AAV vectors. Furthermore, sequences encoding sgRNA(s), and a Cas family nuclease can each be packaged into a separate AAV vector.

Generally, co-expression of a Cas-family enzyme and an autosomal dominant disease-related gene-specific gRNAs in ocular cells, may lead to truncation of the autosomal dominant disease-related gene, thereby preventing the expression of the disease-causing gene.

Codon-modified cDNA of the autosomal dominant disease-related gene may or may not be supplied to ocular cells. The coding sequence of the autosomal dominant disease-related gene may or may not modified in such a way that is resistant to the CRISPR/Cas system. This strategy results in the expression of the autosomal dominant disease-related gene, which can restore or correct the function of the autosomal dominant disease-related gene or fragment after the deletion of endogenous gene(s) or fragments.

The methods of the present disclosure may be applied to various genes, including PDE6A, EFEMP1, mouse Rhodopsin (RHO), and human RHO genes. RP can be caused by autosomal recessive mutations in the PDE6A gene, or autosomal dominant mutations in RHO gene. Mutations in EFEMP1 are responsible for autosomal dominant Malattia Leventinese (ML) and Doyne honeycomb retinal dystrophy (DHRD). Moreover, the methods may be applied to various cell types, including, but not limited to, mouse retina cells as well as human iPS cells. Additionally, the methods described here may be applied in vivo using a mouse model of ocular disease. Thus, methods of the present disclosure can be applied to both animal as well as human subjects.

Furthermore, methods of the present disclosure may be applied to specific gene-humanized mouse model as well as patient-derived cells, allowing for determining the efficiency and efficacy of designed sgRNA and site-specific recombination frequency in human cells, which can be then used as a guide in a clinical setting.

For studies using human and patient-derived cells, AAV2 vector may be used as a backbone vector for the constructs, as it has been shown that AAV2 may transduce human iPS efficiently (Mitsui K et al. Biochem Biophys Res Commun. 2009 Oct. 30; 388(4):711-7; Deyle D R et al. Mol Ther. 2012 January; 20(1):204-13; and Deyle D R et al. Nucleic Acids Res. 2014 March; 42(5):3119-24). However, a variety of other AAV vectors may also be used to carry out the methods of the present disclosure.

The degree of improvement of the autosomal dominant disease by the present methods can vary. For example, the present methods may restore about 20%, about 30%, about 40%, greater than 10%, greater than 20%, greater than 30%, greater than 40%, greater than 50%, greater than 60%, greater than 70%, greater than 80%, or greater than 90%, of the autosomal dominant disease-related gene expression, of the normal levels of the gene product in a control subject, which may be age and sex matched.

In certain embodiments, expression of a wild-type gene (e.g., rhodopsin) can be observed in about 2 weeks following administration to a subject and/or cells. Expression may be maintained for unlimited period of time in nondividing somatic cells (e.g., photoreceptors, neuron cells, muscle cells, etc.). In one embodiment, expression of wild-type rhodopsin is observed in about 3 days, in about 1 week, in about 3 weeks, in about 1 month, in about 2 months, from about 1 week to about 2 weeks, or within different time-frames.

The single-stranded DNA AAV viral vectors have high transduction rates in many different types of cells and tissues. Upon entering the host cells, the AAV genome is converted into double-stranded DNA by host cell DNA polymerase complexes and exist as an episome. In non-dividing host cells, the episomal AAV genome can persist and maintain long-term expression of a therapeutic transgene. (J Virol. 2008 August; 82(16): 7875-7885).

AAV vectors and viral particles of the present disclosure may be employed in various methods and uses. In one embodiment, a method encompasses delivering or transferring a heterologous polynucleotide sequence into a patient or a cell from a patient and includes administering a viral AAV particle, a plurality of AAV viral particles, or a pharmaceutical composition of a AAV viral particle or plurality of AAV viral particles to a patient or a cell of the patient, thereby delivering or transferring a heterologous polynucleotide sequence into the patient or cell of the patient.

In another embodiment, the method is for treating a patient deficient or in need of protein expression or function, or in need of reduced expression or function of an endogenous protein (e.g., an undesirable, aberrant or dysfunctional protein), that includes providing a recombinant AAV viral particle, a plurality of recombinant AAV viral particles, or a pharmaceutical composition of a recombinant AAV viral particle or plurality of AAV viral particles; and administering the recombinant AAV viral particle, plurality of recombinant AAV viral particles, or pharmaceutical composition of AAV viral particle or plurality of AV viral particles to the patient, where the heterologous polynucleotide sequence is expressed in the patient, or wherein the heterologous polynucleotide sequence encodes one or more sgRNA(s) that reduces and or deletes endogenous DNA segment (e.g., an undesirable, aberrant or dysfunctional DNA segment) in the patient.

AAV viral vectors may be selected from among any AAV serotype, including, without limitation, AAV1, AAV2, AAV3, AAV4, AAV5, AAV6, AAV7, AAV8, AAV9, AAV10 or other known and unknown AAV serotypes. In certain embodiment, AAV2 and/or AAV8 are used.

The term AAV covers all subtypes, serotypes and pseudotypes, and both naturally occurring and recombinant forms, except where required otherwise. Pseudotyped AAV refers to an AAV that contains capsid proteins from one serotype and a viral genome of a second serotype.

Additionally, delivery vehicles such as nanoparticle- and lipid-based mRNA or protein delivery systems can be used as an alternative to AAV vectors. Further examples of alternative delivery vehicles include lentiviral vectors, ribonucleoprotein (RNP) complexes, lipid-based delivery system, gene gun, hydrodynamic, electroporation or nucleofection microinjection, and biolistics. Various gene delivery methods are discussed in detail by Nayerossadat et al. (Adv Biomed Res. 2012; 1: 27) and Ibraheem et al. (Int J Pharm. 2014 Jan. 1; 459(1-2):70-83).

Vectors of the present disclosure can comprise any of a number of promoters known to the art, wherein the promoter is constitutive, regulatable or inducible, cell type specific, tissue-specific, or species specific. In addition to the sequence sufficient to direct transcription, a promoter sequence of the invention can also include sequences of other regulatory elements that are involved in modulating transcription (e.g., enhancers, kozak sequences and introns). Many promoter/regulatory sequences useful for driving constitutive expression of a gene are available in the art and include, but are not limited to, for example, CMV (cytomegalovirus promoter), EF1a (human elongation factor 1 alpha promoter), SV40 (simian vacuolating virus 40 promoter), PGK (mammalian phosphoglycerate kinase promoter), Ubc (human ubiquitin C promoter), human beta-actin promoter, rodent beta-actin promoter, CBh (chicken beta-actin promoter), CAG (hybrid promoter contains CMV enhancer, chicken beta actin promoter, and rabbit beta-globin splice acceptor), TRE (Tetracycline response element promoter), H1 (human polymerase III RNA promoter), U6 (human U6 small nuclear promoter), and the like. Moreover, inducible and tissue specific expression of an RNA, transmembrane proteins, or other proteins can be accomplished by placing the nucleic acid encoding such a molecule under the control of an inducible or tissue specific promoter/regulatory sequence. Examples of tissue specific or inducible promoter/regulatory sequences which are useful for this purpose include, but are not limited to, the rhodopsin promoter, the MMTV LTR inducible promoter, the SV40 late enhancer/promoter, synapsin 1 promoter, ET hepatocyte promoter, GS glutamine synthase promoter and many others. Various commercially available ubiquitous as well as tissue-specific promoters can be found at http://www.invivogen.com/prom-a-list. In addition, promoters which are well known in the art can be induced in response to inducing agents such as metals, glucocorticoids, tetracycline, hormones, and the like, are also contemplated for use with the invention. Thus, it will be appreciated that the present disclosure includes the use of any promoter/regulatory sequence known in the art that is capable of driving expression of the desired protein operably linked thereto.

Vectors according to the present disclosure can be transformed, transfected or otherwise introduced into a wide variety of host cells. Transfection refers to the taking up of a vector by a host cell whether or not any coding sequences are in fact expressed. Numerous methods of transfection are known to the ordinarily skilled artisan, for example, lipofectamine, calcium phosphate co-precipitation, electroporation, DEAE-dextran treatment, microinjection, viral infection, and other methods known in the art. Transduction refers to entry of a virus into the cell and expression (e.g., transcription and/or translation) of sequences delivered by the viral vector genome. In the case of a recombinant vector, “transduction” generally refers to entry of the recombinant viral vector into the cell and expression of a nucleic acid of interest delivered by the vector genome.

The present method of treating a disease in a patient can comprise administering to the patient an effective concentration of a composition comprising any of the recombinant AAVs described herein and a pharmaceutically acceptable carrier. In one embodiment, an effective concentration of virus is 1×10⁶-11×10¹³ GC/ml (genome copies/ml). The range of viral concentration effective for the treatment can vary depending on factors including, but not limited to, the specific mutation, patient's age, and other clinical parameters.

Recombinant AAV vectors(s) can be produced in vitro, prior to administration into a patient. Production of recombinant AAV vectors and their use in in vitro and in vivo administration has been discussed in detail by Gray et al. (Curr. Protoc. Neurosci. 2011 October, Chapter: Unit 4.17).

The recombinant AAV containing the desired recombinant DNA can be formulated into a pharmaceutical composition intended for subretinal or intravitreal injection. Such formulation involves the use of a pharmaceutically and/or physiologically acceptable vehicle or carrier, particularly one suitable for administration to the eye, e.g., by subretinal injection, such as buffered saline or other buffers, e.g., HEPES, to maintain pH at appropriate physiological levels, and, optionally, other medicinal agents, pharmaceutical agents, stabilizing agents, buffers, carriers, adjuvants, diluents, etc. For injection, the carrier will typically be a liquid. Exemplary physiologically acceptable carriers include sterile, pyrogen-free water and sterile, pyrogen-free, phosphate buffered saline.

In one embodiment, the carrier is an isotonic sodium chloride solution. In another embodiment, the carrier is balanced salt solution. In one embodiment, the carrier includes Tween. If the virus is to be stored long-term, it may be frozen in the presence of glycerol or Tween-20.

In another embodiment, the pharmaceutically acceptable carrier comprises a surfactant, such as perfluorooctane (Perfluoron liquid). In certain embodiments, the pharmaceutical composition described above is administered to the subject by subretinal injection. In other embodiments, the pharmaceutical composition is administered by intravitreal injection. Other forms of administration that may be useful in the methods described herein include, but are not limited to, direct delivery to a desired organ (e.g., the eye), oral, inhalation, intranasal, intratracheal, intravenous, intramuscular, subcutaneous, intradermal, and other parental routes of administration. Additionally, routes of administration may be combined, if desired.

In certain embodiments, the route of administration is subretinal injection or intravitreal injection.

Administration of the modified AAV or compositions can be effected in one dose, continuously or intermittently throughout the course of treatment. Administration may be through any suitable mode of administration, including but not limited to: intravenous, intra-arterial, intramuscular, intracardiac, intrathecal, subventricular, epidural, intracerebral, intracerebroventricular, sub-retinal, intravitreal, intraarticular, intraocular, intraperitoneal, intrauterine, intradermal, subcutaneous, transdermal, transmuccosal, and inhalation.

Methods of determining the most effective means and dosage of administration are known to those of skill in the art and will vary with the composition used for therapy, the purpose of the therapy and the subject being treated. Single or multiple administrations can be carried out with the dose level and pattern being selected by the treating physician. It is noted that dosage may be impacted by the route of administration. Suitable dosage formulations and methods of administering the agents are known in the art. Non-limiting examples of such suitable dosages may be as low as 1E+9 vector genomes to as much as 1E+17 vector genomes per administration.

This administration of the modified viral particle or compositions of the invention can be done to generate an animal model of the desired disease, disorder, or condition for experimental and screening assays.

The term “about,” as used herein when referring to a numerical value, is meant to encompass variations of 20%, 10%, 5%, 1%, 0.5%, or even 0.1% of the specified amount.

As used herein, “treating” or “treatment” of a disease or a condition in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. In one aspect, the term “treatment” excludes prevention.

The following examples of specific aspects for carrying out the present invention are offered for illustrative purposes only, and are not intended to limit the scope of the present invention in any way.

### Example 1 Self-Terminating Programmable Nuclease Vector

CRISPR-Cas9 system can efficiently modify disease relevant genes in somatic tissues with high efficiency. However, for genome editing applications, persisting expression of nonhuman proteins such as the bacterially derived Cas9 nuclease maybe harmful.

This study used a self-terminating programmable nuclease vector that can terminate the expression of programmable nuclease vector itself after been introduced into the cell.

### Methods

### 1) Plasmid Constructions:

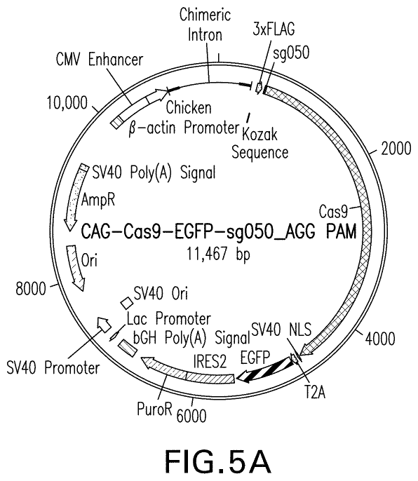

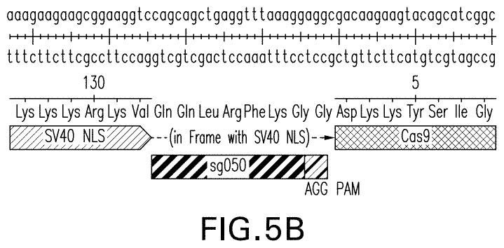

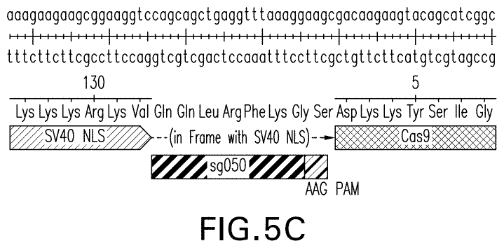

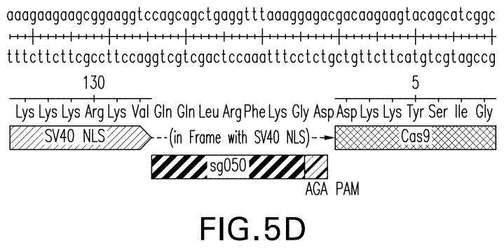

We constructed plasmids of CAG-Cas9-sg050-AGG (canonical PAM), CAG-Cas9-sg050-AAG (suboptimal PAM), and CAG-Cas9-sg050-AGA (suboptimal PAM) which contain the target sequence of human bestrophin 1, flanked by different PAM sites respectively. The plasmids also encode Cas9. See FIGS. 5A-5D.

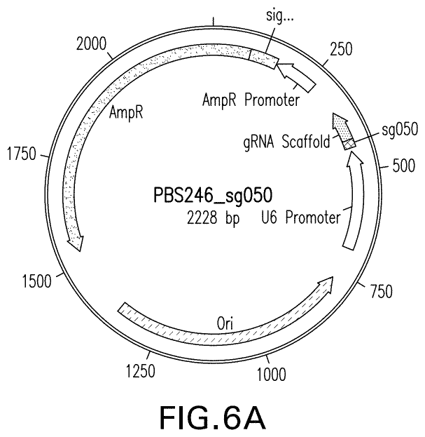

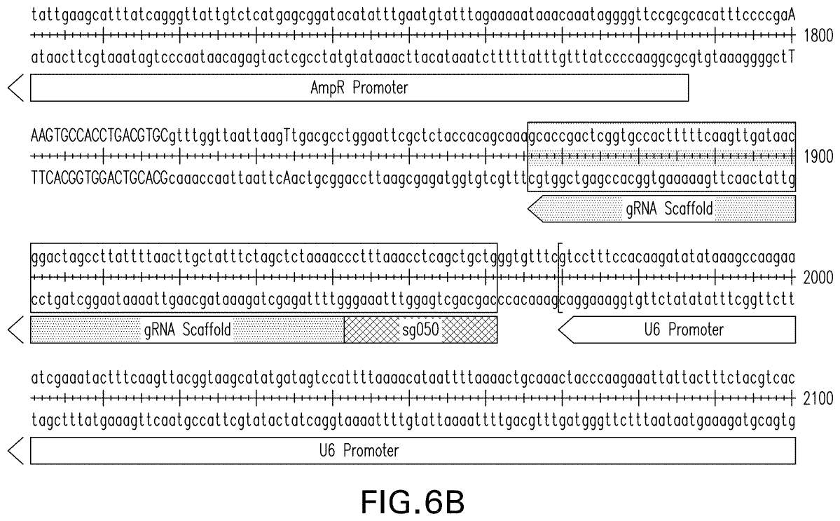

We also constructed PBS246, a plasmid containing the gRNA sequence which targets the human bestrophin 1 gene. See FIGS. 6A-6B.

### 2) Co-Transfection of HEK293 Cells

Plasmids were transfected into HEK293 cell line by Lipofectamine™ 2000 Transfection Reagent (ThermoFisher 11668019).

3) Western Blotting for Cas9 and GFP protein

Antibodies used include: Cell Signaling Cas9 (7A9-3A3) mouse mAb #14697 with dilution factor of 4000×, Abcam Anti-GFP antibody [9F9.F9] (ab1218) with dilution factor of 2000×, secondary antibody Abcam rabbit anti-mouse IgG H&L (HRP) (ab6728).

Cas9 and GFP proteins were detected under three time points of day 3, day 7 and day 14 after transfection.

### Results

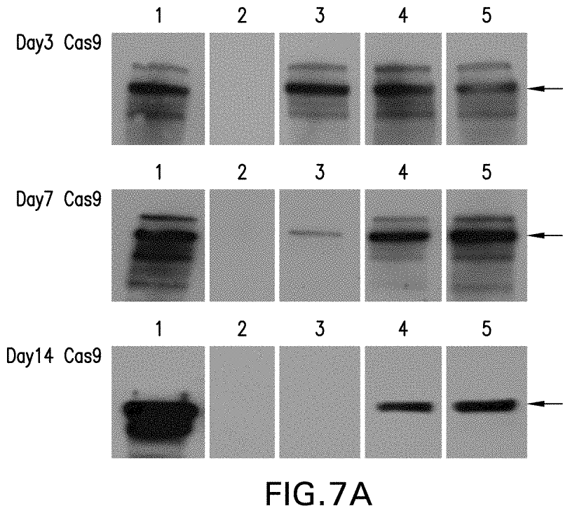

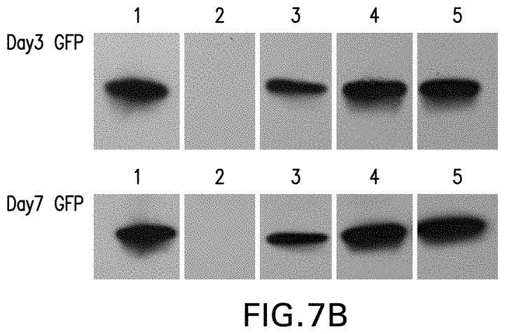

Cas9 western blotting result showed Cas9 protein in the experiment of PBS246-sg050+CAG-Cas9-sg050-AGG decreased on day 7 and completely disappeared at the time point of day 14 (lane 3). This decrease was also observed with the suboptimal PAM constructs CAG-Cas9-sg050-AGA and CAG-Cas9-sg050-AAG, albeit at a less level. This result showed the self-terminating programmable nuclease vector could terminate expression of the Cas9 protein in about 14 days in cell culture. FIG. 7A shows the Cas9 Western blotting results. FIG. 7B shows the GFP Western blotting results.

TABLE 2
Sequences

| Sequence ID Number | Sequence | Species |
|---|---|---|
| SEQ ID NO: 1 (SV40 | 5′ - | Synthetic |
| NLS - target sequence - | CCCAAAGAAGAAGCGGAAGGTCGgacgaagtatccat | |
| Cas9; FIG. 4) | gcagagggaGACAAGAAGTACAGCATCGGCCT -3′ | |
| SEQ ID NO: 2 | 5′ - | Synthetic |
| (complementary to | AGGCCGATGCTGTACTTCTTGTCtccctctgcatggatac | |
| SV40 NLS - target | ttcgtcCGACCTTCCGCTTCTTCTTTGGG - 3′ | |
| sequence - Cas9; FIG. 4) | | |
| SEQ ID NO: 3 (SV40 | Pro Lys Lys Lys Arg Lys Val Gly Arg Ser Ile His Ala | Synthetic |
| NLS - target sequence - | Glu Gly Asp Lys Lys Tyr Ser Ile Gly Leu | |
| Cas9; FIG. 4) | | |
| SEQ ID NO: 4 (target | 5′ - gacgaagtatccatgcagag - 3′ | Homo |
| sequence from | | sapiens |
| rhodopsin (Rho) exon 1; | | |
| FIG. 4) | | |
| SEQ ID NO: 5 | 5′ - | Homo |
| (rhodopsin (Rho) exon 1 | aaattgcatcctgtgggcccgaagacgaagtatccatgcagagaggtgtaga | sapiens |
| including target | gggtgctggtgaagcca - 3′ | |
| sequence; FIG. 4) | | |
| SEQ ID NO: 6 | 5′ - | Homo |
| (complementary to | tggcttcaccagcaccctctacacctctctgcatggatacttcgtcttcgggcc | sapiens |
| rhodopsin (Rho) exon 1 | cacaggatgcaattt - 3′ | |
| including target | | |
| sequence; FIG. 4) | | |
| SEQ ID NO: 7 | Leu Asn Cys Gly Thr Pro Gly Phe Val Phe Tyr Gly His | Homo |
| (rhodopsin (Rho) exon | Leu Ser Thr Tyr Leu Thr Ser Thr Phe Gly | sapiens |
| 1; FIG. 4) | | |
| SEQ ID NO: 8 (SV40 | 5′ - | Synthetic |
| NLS - sg050 target | aaagaagaagcggaaggtccagcagctgaggtttaaaggaggcgacaag | |
| sequence (BEST1) - | aagtacagcatcggc - 3′ | |
| AGG PAM - Cas9; FIG. | | |
| 5B) | | |
| SEQ ID NO: 9 | 5′ - | Synthetic |
| (complementary to | gccgatgctgtacttcttgtcgcctcctttaaacctcagctgctggaccttccgc | |
| SV40 NLS - sg050 | ttcttcttt - 3′ | |
| target sequence | | |
| (BEST1) - AGG PAM - | | |
| Cas9; FIG. 5B) | | |
| SEQ ID NO: 10 (SV40 | Lys Lys Lys Arg Lys Val Gln Gln Leu Arg Phe Lys | Synthetic |
| NLS - sg050 target | Gly Gly Asp Lys Lys Tyr Ser Ile Gly | |
| sequence (BEST1) - | | |
| AGG PAM - Cas9; FIG. | | |
| 5B) | | |
| SEQ ID NO: 11 (sg050 | 5′ - cagcagctgaggtttaaagg - 3′ | Homo |
| target sequence | | sapiens |
| (BEST1); FIGS. 5B-D) | | |
| SEQ ID NO: 12 (SV40 | 5′ - | Synthetic |
| NLS - sg050 target | aaagaagaagcggaaggtccagcagctgaggtttaaaggaagcgacaaga | |
| sequence (BEST1) - | agtacagcatcggc - 3′ | |
| AAG PAM - Cas9; FIG. | | |
| 5C) | | |
| SEQ ID NO: 13 | 5′ - | Synthetic |
| (complementary to | gccgatgctgtacttcttgtcgcttcctttaaacctcagctgctggaccttccgct | |
| SV40 NLS - sg050 | tcttcttt - 3′ | |
| target sequence | | |
| (BEST1) - AAG PAM - | | |
| Cas9; FIG. 5C) | | |
| SEQ ID NO: 14 (SV40 | Lys Lys Lys Arg Lys Val Gln Gln Leu Arg Phe Lys | Synthetic |
| NLS - sg050 target | Gly Ser Asp Lys Lys Tyr Ser Ile Gly | |
| sequence (BEST1) - | | |
| AAG PAM - Cas9; FIG. | | |
| 5C) | | |
| SEQ ID NO: 15 (SV40 | 5′ - | Synthetic |
| NLS - sg050 target | aaagaagaagcggaaggtccagcagctgaggtttaaaggagacgacaaga | |
| sequence (BEST1) - | agtacagcatcggc - 3′ | |
| AGA PAM - Cas9; FIG. | | |
| 5D) | | |
| SEQ ID NO: 16 | 5′ - | Synthetic |
| (complementary to | gccgatgctgtacttcttgtcgtctcctttaaacctcagctgctggaccttccgct | |
| SV40 NLS - sg050 | tcttcttt - 3′ | |
| target sequence | | |
| (BEST1) - AGA PAM - | | |
| Cas9; FIG. 5D) | | |
| SEQ ID NO: 17 (SV40 | Lys Lys Lys Arg Lys Val Gln Gln Leu Arg Phe Lys | Synthetic |
| NLS - sg050 target | Gly Asp Asp Lys Lys Tyr Ser Ile Gly | |
| sequence (BEST1) - | | |
| AGA PAM - Cas9; FIG. | | |
| 5D) | | |
| SEQ ID NO: 18 | 5′ -tattgaagcatttatcagggttattgtctcatgagcggatacatatttgaat | Synthetic |
| (complementary to U6 | gtatttagaaaaataaacaaataggggttccgcgcacatttccccgaA | |
| promoter - sg050 gRNA | AAGTGCCACCTGACGTGCgtttggttaattaagTtgacgcct | |
| for BEST1 - gRNA | ggaattcgctctaccacagcaaagcaccgactcggtgccactttttcaagttg | |
| scaffold - AmpR | ataacggactagccttattttaacttgctatttctagctctaaaaccctttaaacct | |
| promoter; FIG. 6B) | cagctgctgggtgtttcgtcctttccacaagatatataaagccaagaaatcgaa | |
| | atactttcaagttacggtaagcatatgatagtccattttaaaacataattttaaaac | |
| | tgcaaactacccaagaaattattactttctacgtcac - 3′ | |
| SEQ ID NO: 19 (U6 | 5′ - | Synthetic |
| promoter - sg050 gRNA | gtgacgtagaaagtaataatttcttgggtagtttgcagttttaaaattatgttttaa | |
| for BEST1 - gRNA | aatggactatcatatgcttaccgtaacttgaaagtatttcgatttcttggctttatat | |
| scaffold - AmpR | atcttgtggaaaggacgaaacacccagcagctgaggtttaaagggttttaga | |
| promoter; FIG. 6B) | gctagaaatagcaagttaaaataaggctagtccgttatcaacttgaaaaagtg | |
| | gcaccgagtcggtgctttgctgtggtagagcgaattccaggcgtcaActtaa | |
| | ttaaccaaacGCACGTCAGGTGGCACTTTtcggggaaatgt | |
| | gcgcggaacccctatttgtttatttttctaaatacattcaaatatgtatccgctcat | |
| | gagacaataaccctgataaatgcttcaata - 3′ | |
| SEQ ID NO: 20 (gRNA | 5′ - cagcagctgaggtttaaagg - 3′ | Homo |
| for BEST1; FIG. 6B) | | sapiens |
| SEQ ID NO: 21 | GGACGGTGACGTAGAGCGTG | Homo |
| (sgRNA1) | | sapiens |
| SEQ ID NO: 22 | GACGAAGTATCCATGCAGAG | Homo |
| (sgRNA2) | | sapiens |
| SEQ ID NO: 23 | TGAGACCACAAATGAATGCT | Homo |
| (sgRNA-EFEMP1) | | sapiens |
| SEQ ID NO: 24 | AACTACTACACACTCAAGCCTG | Mus |
| (sgRNA-D190N) | | musculus |
| SEQ ID NO: 25 | GTAGTACTGCGGCTGCTCGA | Mus |
| (sgRNA-Rho Exon 1) | | musculus |
| SEQ ID NO: 26 | GCTCATGCTGCCGGCGATTC | Mus |
| (sgRNA-Pde6a^{D670G}) | | musculus |
| SEQ ID NO: 27 | agaggagctg aaacctaccc g | Homo |
| (gRNA1-BEST1) | | sapiens |
| SEQ ID NO: 28 | cacagccaggaatggaccat | Synthetic/ |
| gRNA for BEST1 | | Homo |
| (intron) | | sapiens |
| SEQ ID NO: 29 | atggtccattcctggctgtg | Synthetic/ |
| gRNA for BEST1 | | Homo |
| (intron) | | sapiens |
| SEQ ID NO: 30 | taagtgtgcaagtcagaaca | Synthetic/ |
| gRNA for BEST1 | | Homo |
| (intron) | | sapiens |
| SEQ ID NO: 31 | gtctgaggctgagtatcggg | Synthetic/ |
| gRNA for BEST1 | | Homo |
| (intron) | | sapiens |
| SEQ ID NO: 32 | ggtctcgctatgttgctcag | Synthetic/ |
| gRNA for BEST1 | | Homo |
| (intron) | | sapiens |
| SEQ ID NO: 33 | GGACGGTGACGTAGAGCGTG | Synthetic/ |
| gRNA against RHO | | Homo |
| Exon 1 | | sapiens |
| SEQ ID NO: 34 | GACGAAGTATCCATGCAGAG | Synthetic/ |
| gRNA against RHO | | Homo |
| Exon 1 | | sapiens |

The scope of the present invention is not limited by what has been specifically shown and described hereinabove. Those skilled in the art will recognize that there are suitable alternatives to the depicted examples of materials, configurations, constructions and dimensions. Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety. Variations, modifications and other implementations of what is described herein will occur to those of ordinary skill in the art without departing from the spirit and scope of the invention. While certain embodiments of the present invention have been shown and described, it will be obvious to those skilled in the art that changes and modifications may be made without departing from the spirit and scope of the invention. The matter set forth in the foregoing description is offered by way of illustration only and not as a limitation.

What is claimed is:

1. A polynucleotide comprising:
(a) at least one gene encoding at least one component of an endonuclease system; and
(b) a first target sequence targeted by the endonuclease system.

2. The polynucleotide of claim 1, wherein the at least one gene encodes an RNA-guided DNA endonuclease.

3. The polynucleotide of claim 2, wherein the RNA-guided DNA endonuclease is a Cas enzyme or a variant thereof.

4. The polynucleotide of claim 3, wherein the Cas enzyme is selected from the group consisting of Cas9, Cas nickase and a nuclease-defective Cas (dCas).

5. The polynucleotide of claim 1, wherein the endonuclease system comprises a CRISPR/Cas system.

6. The polynucleotide of claim 1, wherein the at least one gene encodes an RNA selected from the group consisting of a guide RNA (gRNA), a CRISPR RNA (crRNA) and a a single-guide RNA (sgRNA).

7. The polynucleotide of claim 1, wherein the first target sequence is within the at least one gene.

8. The polynucleotide of claim 1, wherein the first target sequence is outside the at least one gene.

9. The polynucleotide of claim 1, wherein the first target sequence is flanked by a first protospacer adjacent motif (PAM).

10. The polynucleotide of claim 9, wherein the first PAM is mutated or suboptimal.

11. The polynucleotide of claim 10, wherein the first PAM comprises a nucleotide sequence NAG or a nucleotide sequence NGA.

12. The polynucleotide of claim 6 wherein there is at least one mismatch between the first target sequence and the gRNA.

13. The polynucleotide of claim 1, wherein the endonuclease system comprises a transcription activator-like effector nuclease (TALEN), a zinc finger nuclease (ZFN), a ZFN dimer, or a ZFNickase.

14. The polynucleotide of claim 1, wherein the first target sequence is derived from a disease-related gene selected from the group consisting of, BEST1, PRDM13, RGR, TEAD1, AIPL1, CRX, GUCA1A, GUCY2D, PITPNM3, PROM1, PRPH2, RIMS1, SEMA4A, UNC119, GNAT1, PDE6B, RHO, WSF1, IMPDH1, OTX2, BEST1, C1QTNF5, CTNNA1, EFEMP1, ELOVL4, FSCN2, GUCA1B, HMCN1, IMPG1, RP1L1, TIMP3, VCAN, MFN2, NR2F1, OPA1, ARL3, CA4, HK1, KLHL7, NR2E3, NRL, PRPF3, PRPF4, PRPF6, PRPF8, PRPF31, RDH12, ROM1, RP1, RP9, RPE65, SNRNP200, SPP2, TOPORS, ABCC6, ATXN7, COL11A1, COL2A1, JAG1, KCNJ13, KIF11, OPA3, PAX2, TREX1, CAPN5, CRB1, FZD4, ITM2B, LRP5, MAPKAPK3, MIR204, OPN1SW, RB1, TSPAN12, and ZNF408.

15. The polynucleotide of claim 1, wherein the first target sequence is derived from a tumor suppressor gene.

16. A composition comprising the polynucleotide of claim 1.

17. A cell comprising the polynucleotide of claim 1.

18. A vector comprising the polynucleotide of claim 1.

19. A method for inactivating an endonuclease system, the method comprising contacting a cell with the polynucleotide of claim 1.

20. A method for modifying gene expression in a cell, the method comprising contacting a cell with the polynucleotide of claim 1.

21. A method of treating an ocular disease in a subject, the method comprising administering the composition of claim 16 to the subject.


SEQUENCE LISTING (34 sequences)

SEQ ID NO: 1 - DNA, 69 bp, Artificial Sequence
  Synthetic
cccaaagaagaagcggaaggtcggacgaagtatccatgcagagggagacaagaagtacagcatcggcct

SEQ ID NO: 2 - DNA, 69 bp, Artificial Sequence
  Synthetic
aggccgatgctgtacttcttgtctccctctgcatggatacttcgtccgaccttccgcttcttctttggg

SEQ ID NO: 3 - protein, 23 aa, Artificial Sequence
  Synthetic
PKKKRKVGRSIHAEGDKKYSIGL

SEQ ID NO: 4 - DNA, 20 bp, Homo Sapiens
gacgaagtatccatgcagag

SEQ ID NO: 5 - DNA, 69 bp, Homo Sapiens
aaattgcatcctgtgggcccgaagacgaagtatccatgcagagaggtgtagagggtgctggtgaagcca

SEQ ID NO: 6 - DNA, 69 bp, Homo Sapiens
tggcttcaccagcaccctctacacctctctgcatggatacttcgtcttcgggcccacaggatgcaattt

SEQ ID NO: 7 - protein, 23 aa, Homo Sapiens
LNCGTPGFVFYGHLSTYLTSTFG

SEQ ID NO: 8 - DNA, 64 bp, Artificial Sequence
  Synthetic
aaagaagaagcggaaggtccagcagctgaggtttaaaggaggcgacaagaagtacagcatcggc

SEQ ID NO: 9 - DNA, 64 bp, Artificial Sequence
  Synthetic
gccgatgctgtacttcttgtcgcctcctttaaacctcagctgctggaccttccgcttcttcttt

SEQ ID NO: 10 - protein, 21 aa, Artificial Sequence
  Synthetic
KKKRKVQQLRFKGGDKKYSIG

SEQ ID NO: 11 - DNA, 20 bp, Homo Sapiens
cagcagctgaggtttaaagg

SEQ ID NO: 12 - DNA, 64 bp, Artificial Sequence
  Synthetic
aaagaagaagcggaaggtccagcagctgaggtttaaaggaagcgacaagaagtacagcatcggc

SEQ ID NO: 13 - DNA, 64 bp, Artificial Sequence
  Synthetic
gccgatgctgtacttcttgtcgcttcctttaaacctcagctgctggaccttccgcttcttcttt

SEQ ID NO: 14 - protein, 21 aa, Artificial Sequence
  Synthetic
KKKRKVQQLRFKGSDKKYSIG

SEQ ID NO: 15 - DNA, 64 bp, Artificial Sequence
  Synthetic
aaagaagaagcggaaggtccagcagctgaggtttaaaggagacgacaagaagtacagcatcggc

SEQ ID NO: 16 - DNA, 64 bp, Artificial Sequence
  Synthetic
gccgatgctgtacttcttgtcgtctcctttaaacctcagctgctggaccttccgcttcttcttt

SEQ ID NO: 17 - protein, 21 aa, Artificial Sequence
  Synthetic
KKKRKVQQLRFKGDDKKYSIG

SEQ ID NO: 18 - DNA, 400 bp, Artificial Sequence
  Synthetic
tattgaagcatttatcagggttattgtctcatgagcggatacatatttgaatgtatttagaaaaataaacaaataggggttccgcgcacatttccccgaaaagtgccacctgacgtgcgtttggttaattaagttgacgcctggaattcgctctaccacagcaaagcaccgactcggtgccactttttcaagttgataacggactagccttattttaacttgctatttctagctctaaaaccctttaaacctcagctgctgggtgtttcgtcctttccacaagatatataaagccaagaaatcgaaatactttcaagttacggtaagcatatgatagtccattttaaaacataattttaaaactgcaaactacccaagaaattattactttctacgtcac

SEQ ID NO: 19 - DNA, 400 bp, Artificial Sequence
  Synthetic
gtgacgtagaaagtaataatttcttgggtagtttgcagttttaaaattatgttttaaaatggactatcatatgcttaccgtaacttgaaagtatttcgatttcttggctttatatatcttgtggaaaggacgaaacacccagcagctgaggtttaaagggttttagagctagaaatagcaagttaaaataaggctagtccgttatcaacttgaaaaagtggcaccgagtcggtgctttgctgtggtagagcgaattccaggcgtcaacttaattaaccaaacgcacgtcaggtggcacttttcggggaaatgtgcgcggaacccctatttgtttatttttctaaatacattcaaatatgtatccgctcatgagacaataaccctgataaatgcttcaata

SEQ ID NO: 20 - DNA, 20 bp, Homo Sapiens
cagcagctgaggtttaaagg

SEQ ID NO: 21 - DNA, 20 bp, Homo Sapiens
ggacggtgacgtagagcgtg

SEQ ID NO: 22 - DNA, 20 bp, Homo Sapiens
gacgaagtatccatgcagag

SEQ ID NO: 23 - DNA, 20 bp, Homo Sapiens
tgagaccacaaatgaatgct

SEQ ID NO: 24 - DNA, 22 bp, Mus Musculus
aactactacacactcaagcctg

SEQ ID NO: 25 - DNA, 20 bp, Mus Musculus
gtagtactgcggctgctcga

SEQ ID NO: 26 - DNA, 20 bp, Mus Musculus
gctcatgctgccggcgattc

SEQ ID NO: 27 - DNA, 21 bp, Homo Sapiens
agaggagctgaaacctacccg

SEQ ID NO: 28 - DNA, 20 bp, Artificial Sequence
  Synthetic
cacagccaggaatggaccat

SEQ ID NO: 29 - DNA, 20 bp, Artificial Sequence
  Synthetic
atggtccattcctggctgtg

SEQ ID NO: 30 - DNA, 20 bp, Artificial Sequence
  Synthetic
taagtgtgcaagtcagaaca

SEQ ID NO: 31 - DNA, 20 bp, Artificial Sequence
  Synthetic
gtctgaggctgagtatcggg

SEQ ID NO: 32 - DNA, 20 bp, Artificial Sequence
  Synthetic
ggtctcgctatgttgctcag

SEQ ID NO: 33 - DNA, 20 bp, Artificial Sequence
  Synthetic
ggacggtgacgtagagcgtg

SEQ ID NO: 34 - DNA, 20 bp, Artificial Sequence
  Synthetic
gacgaagtatccatgcagag